SEQUENCE LISTING (17 sequences)

SEQ ID NO: 1 - DNA, 6044 bp, Homo sapiens
cacagctctgtcctgctctgtgtctttccctgctgctctcaggtcccctgcaggccttggcccctttcctcatctgtagacacacttgagtagcccaggcacagccatgggagattcggagatggcagtctttggggctgccgccccctacctgcgcaagtcagagaaggagcggctagaagcgcagaccaggccttttgacctcaagaaggatgtcttcgtgcctgatgacaaacaggagtttgtcaaggccaagatcgtgtctcgagagggtggcaaagtcactgccgagaccgagtatggcaagacagtgaccgtgaaggaggaccaggtgatgcagcagaacccacccaagttcgacaaaatcgaggacatggccatgctgaccttcctgcatgagcccgcggtgctctacaacctcaaggatcgctacggctcctggatgatctacacctactcgggcctcttctgtgtcaccgtcaacccttacaagtggctgccggtgtacactcctgaggtggtggctgcctaccggggcaagaagaggagcgaggccccgccccacatcttctccatctccgacaacgcctatcagtacatgctgacagacagagaaaaccagtccatcctgatcaccggagaatccggagcagggaagacagtcaacaccaagagggtcatccagtactttgctgttattgcagccattggggaccgcagcaagaaggaccagagcccgggcaagggcaccctggaggaccagatcatccaggccaaccctgctctggaggcctttggcaatgccaagaccgtccggaacgacaactcctcccgcttcgggaaattcattcgaattcattttggggcaacaggaaagttggcatctgcagacatagagacctatcttctggaaaaatccagagttattttccagctgaaagcagagagagattatcacattttctaccaaatcctgtctaacaaaaagcctgagctgctggacatgctgctgatcaccaacaacccctacgattatgcattcatctcccaaggagagaccaccgtggcctccattgatgacgctgaggagctcatggccactgataacgcttttgatgtgctgggcttcacttcagaggagaaaaactccatgtataagctgacaggcgccatcatgcactttggaaacatgaagttcaagctgaagcagcgggaggagcaggcggagccagacggcactgaagaggctgacaagtctgcctacctcatggggctgaactcagccgacctgctcaaggggctgtgccaccctcgggtgaaagtgggcaatgagtacgtcaccaaggggcagaatgtccagcaggtgatatatgccactggggcactggccaaggcagtgtatgagaggatgttcaactggatggtgacgcgcatcaatgccaccctggagaccaagcagccacgccagtacttcataggagtcctggacatcgctggcttcgagatcttcgatttcaacagctttgagcagctctgcatcaacttcaccaacgagaagctgcagcagttcttcaaccaccacatgtttgtgctggagcaggaggagtacaagaaggagggcatcgagtggacattcattgactttggcatggacctgcaggcctgcattgacctcatcgagaagcccatgggcatcatgtccatcctggaagaggagtgcatgttccccaaggccaccgacatgaccttcaaggccaagctgtttgacaaccacctgggcaaatccgccaacttccagaagccacgcaatatcaaggggaagcctgaagcccacttctccctgatccactatgccggcatcgtggactacaacatcattggctggctgcagaagaacaaggatcctctcaatgagactgtcgtgggcttgtatcagaagtcttccctcaagctgctcagcaccctgtttgccaactatgctggggctgatgcgcctattgagaagggcaaaggcaaggccaagaaaggctcgtcctttcagactgtgtcagctctgcacagggaaaatctgaacaagctgatgaccaacttgcgctccacccatccccactttgtacgttgtatcatccctaatgagacaaagtctccaggggtgatggacaaccccctggtcatgcaccagctgcgctgcaatggtgtgctggagggcatccgcatctgcaggaaaggcttccccaaccgcatcctctacggggacttccggcagaggtatcgcatcctgaacccagcggccatccctgagggacagttcattgatagcaggaagggggcagagaagctgctcagctccctggacattgatcacaaccagtacaagtttggccacaccaaggtgttcttcaaggccgggctgctggggctgctggaggaaatgagggacgagaggctgagccgcatcatcacgcgtatccaggcccagtcccgaggtgtgctcgccagaatggagtacaaaaagctgctggaacgtagagactccctgctggtaatccagtggaacattcgggccttcatgggggtcaagaattggccctggatgaagctctacttcaagatcaagccgctgctgaagagtgcagaaagagagaaggagatggcctccatgaaggaggagttcacacgcctcaaagaggcgctagagaagtccgaggctcgccgcaaggagctggaggagaagatggtgtccctgctgcaggagaagaatgacctgcagctccaagtgcaggcggaacaagacaacctggcagatgctgaggagcgctgtgatcagctgatcaaaaacaagattcagctggaggccaaggtgaaggagatgaacgagaggctggaggatgaggaggagatgaatgctgagctcactgccaagaagcgcaagctggaagatgagtgctcagagctcaaaagggacatcgatgatctggagctgacactggccaaagtggagaaggagaaacacgcaacagagaacaaggtgaaaaacctgacagaggagatggctgggctggatgagatcattgccaagctgaccaaggagaagaaagctctgcaagaggcccaccaacaggctctggatgaccttcaggccgaggaggacaaggtcaacaccctgactaaggccaaagtcaagctggagcagcaagtggatgatctggaaggatccctggagcaagagaagaaggtgcgcatggacctggagcgagcgaagcggaagctggagggcgacctgaagctgacccaggagagcatcatggacctggagaatgacaagcagcagctggatgagcggctgaaaaaaaaagactttgagctgaatgctctcaacgcaaggattgaggatgaacaggccctcggcagccagctgcagaagaagctcaaggagcttcaggcacgcatcgaggagctggaggaggagctggaggccgagcgcaccgccagggctaaggtggagaagctgcgctcagacctgtctcgggagctggaggagatcagcgagcggctggaagaggccggcggggccacgtccgtgcagatcgagatgaacaagaagcgcgaggccgagttccagaagatgcggcgggacctggaggaggccacgctgcagcacgaggccactgccgcggccctgcgcaagaagcacgccgacagcgtggccgagctgggcgagcagatcgacaacctgcagcgggtgaagcagaagctggagaaggagaagagcgagttcaagctggagctggatgacgtcacctccaacatggagcagatcatcaaggccaaggctaacctggagaagatgtgccggaccttggaagaccagatgaatgagcaccggagcaaggcggaggagacccagcgttctgtcaacgacctcaccagccagcgggccaagttgcaaaccgagaatggtgagctgtcccggcagctggatgagaaggaggcactgatctcccagctgacccgaggcaagctcacctacacccagcagctggaggacctcaagaggcagctggaggaggaggttaaggcgaagaacgccctggcccacgcactgcagtcggcccggcatgactgcgacctgctgcgggagcagtacgaggaggagacggaggccaaggccgagctgcagcgcgtcctttccaaggccaactcggaggtggcccagtggaggaccaagtatgagacggacgccattcagcggactgaggagctcgaggaggccaagaagaagctggcccagcggctgcaggaagctgaggaggccgtggaggctgttaatgccaagtgctcctcgctggagaagaccaagcaccggctacagaatgagatcgaggacttgatggtggacgtagagcgctccaatgctgctgctgcagccctggacaagaagcagaggaacttcgacaagatcctggccgagtggaagcagaagtatgaggagtcgcagtcggagctggagtcctcgcagaaggaggctcgctccctcagcacagagctcttcaaactcaagaacgcctatgaggagtccctggaacatctggagaccttcaagcgggagaacaaaaacctgcaggaggagatctccgacttgactgagcagttgggttccagcggaaagactatccatgagctggagaaggtccgaaagcagctggaggccgagaagatggagctgcagtcagccctggaggaggccgaggcctccctggagcacgaggagggcaagatcctccgggcccagctggagttcaaccagatcaaggcagagatcgagcggaagctggcagagaaggacgaggagatggaacaggccaagcgcaaccacctgcgggtggtggactcgctgcagacctccctggacgcagagacacgcagccgcaacgaggccctgagggtgaagaagaagatggaaggagacctcaatgagatggagatccagctcagccacgccaaccgcatggccgccgaggcccagaagcaagtcaagagcctccagagcttgttgaaggacacccagattcagctggacgatgcagtccgtgccaacgacgacctgaaggagaacatcgccatcgtggagcggcgcaacaacctgctgcaggctgagctggaggagttgcgtgccgtggtggagcagacagagcggtcccggaagctggcggagcaggagctgattgagactagtgagcgggtgcagctgctgcattcccagaacaccagcctcatcaaccagaagaagaagatggatgctgacctgtcccagctccagactgaagtggaggaggcagtgcaggagtgcaggaatgctgaggagaaggccaagaaggccatcacggatgccgccatgatggcagaggagctgaagaaggagcaggacaccagcgcccacctggagcgcatgaagaagaacatggaacagaccattaaggacctgcagcaccggctggacgaagccgagcagatcgccctcaagggcggcaagaagcagctgcagaagctggaagcgcgggtgcgggagctggagaatgagctggaggccgagcagaagcgcaacgcagagtcggtgaagggcatgaggaagagcgagcggcgcatcaaggagctcacctaccagacggaggaggacaggaaaaacctgctgcggctgcaggacctggtagacaagctgcagctaaaggtcaaggcctacaagcgccaggccgaggaggcggaggagcaagccaacaccaacctgtccaagttccgcaaggtgcagcacgagctggatgaggcagaggagcgggcggacatcgccgagtcccaggtcaacaagctgcgggccaagagccgtgacattggcacgaagggcttgaatgaggagtagctttgccacatcttgatctgctcagccctggaggtgccagcaaagccccatgctggagcctgtgtaacagctccttgggaggaagcagaataaagcaattttccttgaagccgagaaaaaaaaaaaaaaa

SEQ ID NO: 2 - DNA, 5937 bp, Homo sapiens
agagagactcctgcggcccagattcttcaggattctccgtgaagggataaccaggggaagcaccaagatgaccgatgcccagatggctgactttggggcagcggcccagtacctccgcaagtcagagaaggagcgtctagaggcccagacccggccctttgacattcgcactgagtgcttcgtgcccgatgacaaggaagagtttgtcaaagccaagattttgtcccgggagggaggcaaggtcattgctgaaaccgagaatgggaagacggtgactgtgaaggaggaccaggtgttgcagcagaacccacccaagttcgacaagattgaggacatggccatgctgaccttcctgcacgagcccgcggtgcttttcaacctcaaggagcgctacgcggcctggatgatatatacctactcgggcctcttctgtgtcactgtcaacccctacaagtggctgccggtgtacaatgccgaggtggtggccgcctaccggggcaagaagaggagtgaggccccgccccacatcttctccatctccgacaacgcctatcagtacatgctgacagatcgggagaaccagtccatcctcatcacgggagaatccggggcggggaagactgtgaacaccaagcgtgtcatccagtactttgccagcattgcagccataggtgaccgtggcaagaaggacaatgccaatgcgaacaagggcaccctggaggaccagatcatccaggccaaccccgctctggaggccttcggcaatgccaagactgtccggaacgacaactcctcccgctttgggaaattcattaggatccactttggggccactggaaagctggcttctgcagacatagagacctacctgctggagaagtcccgggtgatcttccagctgaaagctgagagaaactaccacatcttctaccagattctgtccaacaagaagccggagttgctggacatgctgctggtcaccaacaatccctacgactacgccttcgtgtctcagggagaggtgtccgtggcctccattgatgactccgaggagctcatggccaccgatagtgcctttgacgtgctgggcttcacttcagaggagaaagctggcgtctacaagctgacgggagccatcatgcactacgggaacatgaagttcaagcagaagcagcgggaggagcaggcggagccagacggcaccgaagatgctgacaagtcggcctacctcatggggctgaactcagctgacctgctcaaggggctgtgccaccctcgggtgaaagtgggcaacgagtatgtcaccaaggggcagagcgtgcagcaggtgtactactccatcggggctctggccaaggcagtgtatgagaagatgttcaactggatggtgacgcgcatcaacgccaccctggagaccaagcagccacgccagtacttcataggagtcctggacatcgctggcttcgagatcttcgacttcaacagctttgagcagctctgcatcaacttcaccaacgagaagctgcagcagttcttcaaccaccacatgttcgtgctggagcaggaggagtacaagaaggagggcattgagtggacattcattgactttggcatggacctgcaggcctgcattgacctcatcgagaagcccatgggcatcatgtccatcctggaggaggagtgcatgttccccaaggccactgacatgaccttcaaggccaagctgtacgacaaccacctgggcaagtccaacaatttccagaagccacgcaacatcaaggggaagcaggaagcccacttctccctgatccactacgccggcactgtggactacaacatcctgggctggctggaaaaaaacaaggatcctctcaacgagactgttgtggccctgtaccagaagtcctccctcaagctcatggccactctcttctcctcctacgcaactgccgatactggggacagtggtaaaagcaaaggaggcaagaaaaagggctcatccttccagacggtgtcggctctccaccgggaaaatctcaacaagctaatgaccaacctgaggaccacccatcctcactttgtgcgttgcatcatccccaatgagcggaaggctccaggggtgatggacaaccccctggtcatgcaccagctgcgctgcaatggcgtgctggagggcatccgcatctgcaggaagggcttccccaaccgcatcctctacggggacttccggcagaggtatcgcatcctgaacccagtggccatccctgagggacagttcattgatagcaggaaggggacagagaagctgctcagctctctggacattgatcacaaccagtacaagtttggccacaccaaggtgttcttcaaggcagggctgcttgggctgctggaggagatgcgggatgagaggctgagccgcatcatcacgcgcatgcaggcccaagcccggggccagctcatgcgcattgagttcaagaagatagtggaacgcagggatgccctgctggtaatccagtggaacattcgggccttcatgggggtcaagaattggccctggatgaagctctacttcaagatcaagccgctgctgaagagcgcagagacggagaaggagatggccaccatgaaggaagagttcgggcgcatcaaagagacgctggagaagtccgaggctcgccgcaaggagctggaggagaagatggtgtccctgctgcaggagaagaatgacctgcagctccaagtgcaggcggaacaagacaacctcaatgatgctgaggagcgctgcgaccagctgatcaaaaacaagattcagctggaggccaaagtaaaggagatgaatgagaggctggaggatgaggaggagatgaacgcggagctcactgccaagaagcgcaagctggaagacgagtgctcagagctcaagaaggacattgatgacctggagctgacactggccaaggtggagaaggagaagcatgcaacagagaacaaggtgaagaacctaacagaggagatggctgggctggatgaaatcatcgctaagctgaccaaggagaagaaagctctacaagaggcccatcagcaggccctggatgaccttcaggttgaggaagacaaggtcaacagcctgtccaagtctaaggtcaagctggagcagcaggtggatgatctggagggatccctagagcaagagaagaaggtgcgcatggacctggagcgagcaaagcggaaactggagggcgacctgaagctgacccaggagagcatcatggacctggaaaatgataaactgcagctggaagaaaagcttaagaagaaggagtttgacattaatcagcagaacagtaagattgaggatgagcaggtgctggcccttcaactacagaagaaactgaaggaaaaccaggcacgcatcgaggagctggaggaggagctggaggccgagcgcaccgccagggctaaggtggagaagctgcgctcagacctgtctcgggagctggaggagatcagcgagcggctggaagaggccggcggggccacgtccgtgcagatcgagatgaacaagaagcgcgaggccgagttccagaagatgcggcgggacctggaggaggccacgctgcagcacgaggccactgccgcggccctgcgcaagaagcacgccgacagcgtggccgagctgggcgagcagatcgacaacctgcagcgggtgaagcagaagctggagaaggagaagagcgagttcaagctggagctggatgacgtcacctccaacatggagcagatcatcaaggccaaggcaaacctggagaaagtgtctcggacgctggaggaccaggccaatgagtaccgcgtgaagctagaagaggcccaacgctccctcaatgatttcaccacccagcgagccaagctgcagaccgagaatggagagttggcccggcagctagaggaaaaggaggcgctaatctcgcagctgacccgggggaagctctcttatacccagcaaatggaggacctcaaaaggcagctggaggaggagggcaaggcgaagaacgccctggcccatgcactgcagtcggcccggcatgactgcgacctgctgcgggagcagtacgaggaggagacagaggccaaggccgagctgcagcgcgtcctgtccaaggccaactcggaggtggcccagtggaggaccaagtatgagacggacgccattcagcggactgaggagctcgaagaggccaaaaagaagctggcccagcggctgcaggatgccgaggaggccgtggaggctgttaatgccaagtgctcctcactggagaagaccaagcaccggctacagaatgagatagaggacttgatggtggacgtagagcgctccaatgctgctgctgcagccctggacaagaagcagagaaactttgacaagatcctggccgagtggaagcagaagtatgaggagtcgcagtctgagctggagtcctcacagaaggaggctcgctccctcagcacagagctcttcaagctcaagaacgcctacgaggagtccctggagcacctagagaccttcaagcgggagaacaagaaccttcaggaggaaatctcggaccttactgagcagctaggagaaggaggaaagaatgtgcatgagctggagaaggtccgcaaacagctggaggtggagaagctggagctgcagtcagccctggaggaggcagaggcctccctggagcacgaggagggcaagatcctccgggcccagctagagttcaaccagatcaaggcagagatcgagcggaagctggcagagaaggacgaggagatggaacaggccaagcgcaaccaccagcgggtggtggactcgctgcagacctccctggatgcagagacacgcagccgcaacgaggtcctgagggtgaagaagaagatggaaggagacctcaatgagatggagatccagctcagccacgccaaccgcatggctgccgaggcccagaagcaagtcaagagcctccagagcttgctgaaggacacccagatccagctggacgatgcggtccgtgccaacgacgacctgaaggagaacatcgccatcgtggagcggcgcaacaacctgctgcaggctgagctggaggagctgcgtgccgtggtggagcagacagagcggtcccggaagctggcggagcaggagctgattgagaccagcgagcgggtgcagctgctgcattcccagaacaccagcctcatcaaccagaagaagaagatggagtcggatctgacccagctccagtcggaagtggaggaggcagtgcaggagtgcagaaacgccgaggagaaggccaagaaggccatcacggatgccgccatgatggcagaggagctgaagaaggagcaggacaccagcgcccacctggagcgcatgaagaagaacatggagcagaccattaaggacctgcagcaccggctggacgaggccgagcagatcgccctcaagggaggcaagaagcagctgcagaagctggaagcgcgggtgcgggagctggagggtgagctggaggccgagcagaagcgcaacgcagagtcggtgaagggcatgaggaagagcgagcggcgcatcaaggagctcacctaccagacagaggaagacaaaaagaacctgctgcggctacaggacctggtggacaagctgcaactgaaggtcaaggcctacaagcgccaggccgaggaggcggaggagcaagccaacaccaacctgtccaagttccgcaaggtgcagcatgagctggatgaggcagaggagcgggcggacatcgctgagtcccaggtcaacaagcttcgagccaagagccgtgacattggtgccaagcaaaaaatgcacgatgaggagtgacactgcctcgggaacctcactcttgccaacctgtaataaatatgagtgcc

SEQ ID NO: 3 - DNA, 1153 bp, Homo sapiens
ccccgctgagactgagcagacgcctccaggatctgtcggcagctgctgttctgagggagagcagagaccatgtctgacatagaagaggtggtggaagagtacgaggaggaggagcaggaagaagcagctgttgaagaagaggaggactggagagaggacgaagacgagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgatcgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaagacagagcggaaaagtgggaagaggcagactgagcgggaaaagaagaagaagattctggctgagaggaggaaggtgctggccattgaccacctgaatgaagatcagctgagggagaaggccaaggagctgtggcagagcatctataacttggaggcagagaagttcgacctgcaggagaagttcaagcagcagaaatatgagatcaatgttctccgaaacaggatcaacgataaccagaaagtctccaagacccgcgggaaggctaaagtcaccgggcgctggaaatagagcctggcctccttcaccaaagatctgctcctcgctcgcacctgcctccggcctgcactcccccagttcccgggccctcctgggcaccccaggcagctcctgtttggaaatggggagctggcctaggtgggagccaccactcctgcctgcccccacacccactccacaccagtaataaaaagccaccacacactga

SEQ ID NO: 4 - DNA, 1132 bp, Homo sapiens
ccccgctgagactgagcagacgcctccaggatctgtcggcagctgctgttctgagggagagcagagaccatgtctgacatagaagaggtggtggaagagtacgaggaggaggagcaggaagaagcagctgttgaagagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgatcgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaagcaggcccagacagagcggaaaagtgggaagaggcagactgagcgggaaaagaagaagaagattctggctgagaggaggaaggtgctggccattgaccacctgaatgaagatcagctgagggagaaggccaaggagctgtggcagagcatctataacttggaggcagagaagttcgacctgcaggagaagttcaagcagcagaaatatgagatcaatgttctccgaaacaggatcaacgataaccagaaagtctccaagacccgcgggaaggctaaagtcaccgggcgctggaaatagagcctggcctccttcaccaaagatctgctcctcgctcgcacctgcctccggcctgcactcccccagttcccgggccctcctgggcaccccaggcagctcctgtttggaaatggggagctggcctaggtgggagccaccactcctgcctgcccccacacccactccacaccagtaataaaaagccaccacacactga

SEQ ID NO: 5 - DNA, 1123 bp, Homo sapiens
ccccgctgagactgagcagacgcctccaggatctgtcggcagctgctgttctgagggagagcagagaccatgtctgacatagaagaggtggtggaagagtacgaggaggaggagcaggaagaagcagctgttgaagagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgatcgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaagacagagcggaaaagtgggaagaggcagactgagcgggaaaagaagaagaagattctggctgagaggaggaaggtgctggccattgaccacctgaatgaagatcagctgagggagaaggccaaggagctgtggcagagcatctataacttggaggcagagaagttcgacctgcaggagaagttcaagcagcagaaatatgagatcaatgttctccgaaacaggatcaacgataaccagaaagtctccaagacccgcgggaaggctaaagtcaccgggcgctggaaatagagcctggcctccttcaccaaagatctgctcctcgctcgcacctgcctccggcctgcactcccccagttcccgggccctcctgggcaccccaggcagctcctgtttggaaatggggagctggcctaggtgggagccaccactcctgcctgcccccacacccactccacaccagtaataaaaagccaccacacactga

SEQ ID NO: 6 - DNA, 1114 bp, Homo sapiens
ccccgctgagactgagcagacgcctccaggatctgtcggcagctgctgttctgagggagagcagagaccatgtctgacatagaagaggtggtggaagagtacgaggaggaggagcaggaagagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgatcgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaaggcccagacagagcggaaaagtgggaagaggcagactgagcgggaaaagaagaagaagattctggctgagaggaggaaggtgctggccattgaccacctgaatgaagatcagctgagggagaaggccaaggagctgtggcagagcatctataacttggaggcagagaagttcgacctgcaggagaagttcaagcagcagaaatatgagatcaatgttctccgaaacaggatcaacgataaccagaaagtctccaagacccgcgggaaggctaaagtcaccgggcgctggaaatagagcctggcctccttcaccaaagatctgctcctcgctcgcacctgcctccggcctgcactcccccagttcccgggccctcctgggcaccccaggcagctcctgtttggaaatggggagctggcctaggtgggagccaccactcctgcctgcccccacacccactccacaccagtaataaaaagccaccacacactga

SEQ ID NO: 7 - DNA, 1585 bp, Homo sapiens
gcctggtcccgcctctcctgccccttgtgctcagcgctacctgctgcccggacacatccagagctggccgacgggtgcgcgggcgggcggcggcaccatgcagggaagctgccaggggccgtgggcagcgccgctttctgccgcccacctggcgctgtgagactggcgctgccaccatgttccccagccctgctctcacgcccacgcccttctcagtcaaagacatcctaaacctggaacagcagcagcgcagcctggctgccgccggagagctctctgcccgcctggaggcgaccctggcgccctcctcctgcatgctggccgccttcaagccagaggcctacgctgggcccgaggcggctgcgccgggcctcccagagctgcgcgcagagctgggccgcgcgccttcaccggccaagtgtgcgtctgcctttcccgccgcccccgccttctatccacgtgcctacagcgaccccgacccagccaaggaccctagagccgaaaagaaagagctgtgcgcgctgcagaaggcggtggagctggagaagacagaggcggacaacgcggagcggccccgggcgcgacggcggaggaagccgcgcgtgctcttctcgcaggcgcaggtctatgagctggagcggcgcttcaagcagcagcggtacctgtcggcccccgaacgcgaccagctggccagcgtgctgaaactcacgtccacgcaggtcaagatctggttccagaaccggcgctacaagtgcaagcggcagcggcaggaccagactctggagctggtggggctgcccccgccgccgccgccgcctgcccgcaggatcgcggtgccagtgctggtgcgcgatggcaagccatgcctaggggactcggcgccctacgcgcctgcctacggcgtgggcctcaatccctacggttataacgcctaccccgcctatccgggttacggcggcgcggcctgcagccctggctacagctgcactgccgcttaccccgccgggccttccccagcgcagccggccactgccgccgccaacaacaacttcgtgaacttcggcgtcggggacttgaatgcggttcagagccccgggattccgcagagcaactcgggagtgtccacgctgcatggtatccgagcctggtagggaagggacccgcgtggcgcgaccctgaccgatcccacctcaacagctccctgactctcggggggagaaggggctcccaacatgaccctgagtcccctggattttgcattcactcctgcggagacctaggaactttttctgtcccacgcgcgtttgttcttgcgcacgggagagtttgtggcggcgattatgcagcgtgcaatgagtgatcctgcagcctggtgtcttagctgtccccccaggagtgccctccgagagtccatgggcacccccggttggaactgggactgagctcgggcacgcagggcctgagatctggccgcccattccgcgagccagggccgggcgcccgggcctttgctatctcgccgtcgcccgcccacgcacccacccgtatttatgtttttacctattgctgtaagaaatgacgatcc

SEQ ID NO: 8 - DNA, 3372 bp, Homo sapiens
gtcgccgctgcagctccgggggctcccaggggagcgtgcgcggaacctccaggcccagcaggaccccggctgcggcgaggaggaaggagccagcctagcagcttctgcgcctgtggccgcgggtgtcctggaggcctctcggtgtgacgagtgggggacccgaaggctcgtgcgccacctccaggcctggacgctgccctccgtcttctgcccccaataggtgcgccggaccttcaggccctggggtgaattcagctgctcctacatcagcttccggaaccaccaaaaattcaaattgggattttccggagtaaacaagagcctagagccctttgctcaatgctggatttaatacgtatatatttttaagcgagttggttttttcccctttgatttttgatcttcgcgacagttcctcccacgcatattatcgttgttgccgtcgttttctctccccgcgtggctccttgacctgcgagggagagagaggacaccgaagccgggagctcgcagggaccatgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcgatatgtttgacgacttctcagaaggcagagagtgtgtcaactgtggggctatgtccaccccgctctggaggcgagatgggacgggtcactatctgtgcaacgcctgcggcctctaccacaagatgaacggcatcaaccggccgctcatcaagcctcagcgccggctgtccgcctcccgccgagtgggcctctcctgtgccaactgccagaccaccaccaccacgctgtggcgccgcaatgcggagggcgagcctgtgtgcaatgcctgcggcctctacatgaagctccacggggtacccaggcctcttgcaatgcggaaagaggggatccaaaccagaaaacggaagcccaagaacctgaataaatctaagacaccagcagctccttcaggcagtgagagccttcctcccgccagcggtgcttccagcaactccagcaacgccaccaccagcagcagcgaggagatgcgtcccatcaagacggagcctggcctgtcatctcactacgggcacagcagctccgtgtcccagacgttctcagtcagtgcgatgtctggccatgggccctccatccaccctgtcctctcggccctgaagctctccccacaaggctatgcgtctcccgtcagccagtctccacagaccagctccaagcaggactcttggaacagcctggtcttggccgacagtcacggggacataatcactgcgtaatcttccctcttccctcctcaaattcctgcacggacctgggacttggaggatagcaaagaaggaggccctgggctcccaggggccggcctcctctgcctggtaatgactccagaacaacaactgggaagaaacttgaagtcgacaatctggttaggggaagcgggtgttggattttctcagatgcctttacacgctgatgggactggagggagcccacccttcagcacgagcacactgcatctctcctgtgagttggagacttctttcccaagatgtccttgtcccctgcgttccccactgtggcctagaccgtgggttttgcattgtgtttctagcaccgaggatctgagaacaagcggagggccgggccctgggacccctgctccagcccgaatgacggcatctgtttgccatgtacctggatgcgacgggcccctggggacaggcccttgccccatccatccgcttgaggcatggcaccgccctgcatccctaataccaaatctgactccaaaattgtggggtgtgacatacaagtgactgaacacttcctggggagctacaggggcacttaacccaccacagcacagcctcatcaaaatgcagctggcaacttctcccccaggtgccttccccctgctgccggcctttgctccttcacttccaacatctctcaaaataaaaatccctcttcccgctctgagcgattcagctctgcccgcagcttgtacatgtctctcccctggcaaaacaagagctgggtagtttagccaaacggcaccccctcgagttcactgcagacccttcgttcaccgtgtcacacatagaggggttctgagtaagaacaaaacgttctgctgctcaagccagtctggcaagcactcagcccagcctcgaggtccttctggggagagtgtaagtggacagagtcctggtcagggggcaggagtgtcccaagggctggcccacctgctgtctgtctgctcctcctagcccttggtcagatggcagccagagtccctcaggacctgcagcctcgccccggcagaagtcttttgtccaggaggcaaaaagccagagattctgcaacacgaattcgaagcaaacaaacacaacacaacagaattcctggaaagaagacgactgctaagacacggcaggggggcctggagggagcctccgactctgagctgctccgggatctgccgcgttctcctctgcacattgctgtttctgcccctgatgctggagctcaaggagactccttcctctttctcagcagagctgtagctgactgtggcattactacgcctccccacacgcccagacccctcactccaaaatcctactggctgtagcagagaatacctttgaaccaagattctgttttaatcatcatttacattgttttcttccaaaggccccctcgtataccctccctaacccacaaacctgttaacattgtcttaaggtgaaatggctggaaaatcagtatttaactaataaatttatctgtattcctctt

SEQ ID NO: 9 - DNA, 840 bp, Homo sapiens
agggacagacgtaggccaagagaggggaaccagagaggaaccagaggggagagacagagcagcaagcagtggattgctccttgacgacgccagcatgagctccttctccaccaccaccgtgagcttcctccttttactggcattccagctcctaggtcagaccagagctaatcccatgtacaatgccgtgtccaacgcagacctgatggatttcaagaatttgctggaccatttggaagaaaagatgcctttagaagatgaggtcgtgcccccacaagtgctcagtgagccgaatgaagaagcgggggctgctctcagccccctccctgaggtgcctccctggaccggggaagtcagcccagcccagagagatggaggtgccctcgggcggggcccctgggactcctctgatcgatctgccctcctaaaaagcaagctgagggcgctgctcactgcccctcggagcctgcggagatccagctgcttcgggggcaggatggacaggattggagcccagagcggactgggctgtaacagcttccggtactgaagataacagccagggaggacaagcagggctgggcctagggacagactgcaagaggctcctgtcccctggggtctctgctgcatttgtgtcatcttgttgccatggagttgtgatcatcccatctaagctgcagcttcctgtcaacacttctcacatcttatgctaactgtagataaagtggtttgatggtgacttcctcgcctctcccaccccatgcattaaattttaaggtagaacctcacctgttactgaaagtggtttgaaagtgaataaacttcagcaccatggac

SEQ ID NO: 10 - DNA, 708 bp, Homo sapiens
ccccgcaggctgagggcaggtgggaagcaaacccggacgcatcgcagcagcagcagcagcagcagaagcagcagcagcagcctccgcagtccctccagagacatggatccccagacagcaccttcccgggcgctcctgctcctgctcttcttgcatctggctttcctgggaggtcgttcccacccgctgggcagccccggttcagcctcggacttggaaacgtccgggttacaggagcagcgcaaccatttgcagggcaaactgtcggagctgcaggtggagcagacatccctggagcccctccaggagagcccccgtcccacaggtgtctggaagtcccgggaggtagccaccgagggcatccgtgggcaccgcaaaatggtcctctacaccctgcgggcaccacgaagccccaagatggtgcaagggtctggctgctttgggaggaagatggaccggatcagctcctccagtggcctgggctgcaaagtgctgaggcggcattaagaggaagtcctggctgcagacacctgcttctgattccacaaggggctttttcctcaaccctgtggccgcctttgaagtgactcatttttttaatgtatttatgtatttatttgattgttttatataagatggtttcttacctttgagcacaaaatttccacggtgaaataaagtcaacattataagctttaaaaaaaaaaa

SEQ ID NO: 11 - DNA, 3720 bp, Homo sapiens
aggatacagcggcttctgcgcgacttataagagctccttgtgcggcgccattttaagcctctcggtctgtggcagcagcgttggcccggccccgggagcggagagcgaggggaggcggagacggaggaaggtctgaggagcagcttcagtccccgccgagccgccaccgcaggtcgaggacggtcggactcccgcggcgggaggagcctgttcccctgagggtatttgaagtataccatacaactgttttgaaaatccagcgtggacaatggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttaggtaagaagttttaaaaagccagtttgggtaaaatacttttactctgcctacagaacttcagaaagacttggttggtagggtgggagtggtttaggctatttgtaaatctgccacaaaaacaggtatatactttgaaaggagatgtcttggaacattggaatgttctcagatttctggttgttatgtgatcatgtgtggaagttattaactttaatgttttttgccacagcttttgcaacttaatactcaaatgagtaacatttgctgttttaaacattaatagcagcctttctctctttatacagctgtattgtctgaacttgcattgtgattggcctgtagagttgctgagagggctcgaggggtgggctggtatctcagaaagtgcctgacacactaaccaagctgagtttcctatgggaacaattgaagtaaactttttgttctggtcctttttggtcgaggagtaacaatacaaatggattttgggagtgactcaagaagtgaagaatgcacaagaatggatcacaagatggaatttatcaaaccctagccttgcttgttaaatttttttttttttttttttaagaatatctgtaatggtactgactttgcttgctttgaagtagctctttttttttttttttttttttttttgcagtaactgttttttaagtctctcgtagtgttaagttatagtgaatactgctacagcaatttctaatttttaagaattgagtaatggtgtagaacactaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcattcgattg

SEQ ID NO: 12 - DNA, 1411 bp, Homo sapiens
ccttcgcaagccctcatttcaccaggcccccggcttggggcgccttccttccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 13 - DNA, 1155 bp, Homo sapiens
catgagtcagtgaacagggaatgggtgaatgacatttgtgggtaggttatttctagaagttaggtgggcagcttggaaggcagatgcacttctacagactattccttggggccacacgtaggttcttgaatcccgaatggaaaggggagattgataactggtgtgtttatgttcttacaagtcttctgccttttaaaatccagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 14 - DNA, 2098 bp, Homo sapiens
attataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtgaagatgagtgaaactgatattactcaatttcagtctggacactggctgaatccttcctctcccctcctcccatccctcataggatttttcttgtttggaaaccacgtgttctggtttccatgatgcccatccagtcaatctcatggagggtggagtatggttggagcctaatcagcgaggtttctttttttttttttttcctattggatcttcctggagaaaatactttttttttttttttttttgaaacggagtcttgctctgtcgcccaggctggagtgcagtggcgcggtcttggctcactgcaagctccgtctcccgggttcacgccattctcctgcctcagcctcccgagcagctgggactacaggcgcccgccacctcgcccggctaatattttgtatttttagtagagacggggtttcactgtgttagccaggatggtctcgatctcctgaccttgtgatccacccgcctcggcctccctaacagctgggatttacaggcgtgagccaccgcgccctgcctagaaaagacattttaataaccttggctgccgtctctggctatagataagtagatctaatactagtttggatatctttagggtttagaatctaacctcaagaataagaaatacaagtacaaattggtgatgaagatgtattcgtattgtttgggattgggaggctttgcttattttttaaaaactattgaggtaaagggttaagctgtaacatacttaattgatttcttaccgtttttggctctgttttgctatatcccctaatttgttggttgtgctaatctttgtagaaagaggtctcgtatttgctgcatcgtaatgacatgagtactgctttagttggtttaagttcaaatgaatgaaacaactatttttcctttagttgattttaccctgatttcaccgagtgtttcaatgagtaaatatacagcttaaacat

SEQ ID NO: 15 - RNA, 1871 nt, Homo sapiens
uaccugguugauccugccaguagcauaugcuugucucaaagauuaagccaugcaugucuaaguacgcacggccgguacagugaaacugcgaauggcucauuaaaucaguuaugguuccuuuggucgcucgcuccucuccuacuuggauaacugugguaauucuagagcuaauacaugccgacgggcgcugacccccuucgcgggggggaugcgugcauuuaucagaucaaaaccaacccggucagccccucuccggccccggccggggggcgggcgccggcggcuuuggugacucuagauaaccucgggccgaucgcacgccccccguggcggcgacgacccauucgaacgucugcccuaucaacuuucgaugguagucgccgugccuaccauggugaccacgggugacggggaaucaggguucgauuccggagagggagccugagaaacggcuaccacauccaaggaaggcagcaggcgcgcaaauuacccacucccgacccggggagguagugacgaaaaauaacaauacaggacucuuucgaggcccuguaauuggaaugaguccacuuuaaauccuuuaacgaggauccauuggagggcaagucuggugccagcagccgcgguaauuccagcuccaauagcguauauuaaaguugcugcaguuaaaaagcucguaguuggaucuugggagcgggcgggcgguccgccgcgaggcgagccaccgcccguccccgccccuugccucucggcgcccccucgaugcucuuagcugagugucccgcggggcccgaagcguuuacuuugaaaaaauuagaguguucaaagcaggcccgagccgccuggauaccgcagcuaggaauaauggaauaggaccgcgguucuauuuuguugguuuucggaacugaggccaugauuaagagggacggccgggggcauucguauugcgccgcuagaggugaaauucuuggaccggcgcaagacggaccagagcgaaagcauuugccaagaauguuuucauuaaucaagaacgaaagucggagguucgaagacgaucagauaccgucguaguuccgaccauaaacgaugccgaccggcgaugcggcggcguuauucccaugacccgccgggcagcuuccgggaaaccaaagucuuuggguuccggggggaguaugguugcaaagcugaaacuuaaaggaauugacggaagggcaccaccaggaguggagccugcggcuuaauuugacucaacacgggaaaccucacccggcccggacacggacaggauugacagauugauagcucuuucucgauuccgugggugguggugcauggccguucuuaguugguggagcgauuugucugguuaauuccgauaacgaacgagacucuggcaugcuaacuaguuacgcgacccccgagcggucggcgucccccaacuucuuagagggacaaguggcguucagccacccgagauugagcaauaacaggucugugaugcccuuagauguccggggcugcacgcgcgcuacacugacuggcucagcgugugccuacccuacgccggcaggcgcggguaacccguugaaccccauucgugauggggaucggggauugcaauuauuccccaugaacgagggaauucccgaguaagugcgggucauaagcuugcguugauuaagucccugcccuuuguacacaccgcccgucgcuacuaccgauuggaugguuuagugaggcccucggaucggccccgccggggucggcccacggcccuggcggagcgcugagaagacggucgaacuugacuaucuagaggaaguaaaagucguaacaagguuuccguaggugaaccugcggaaggaucauua

SEQ ID NO: 16 - DNA, 1288 bp, Coturnix coturnix
aagccggactcagccctcgctctaccccgggtaaaggttgtgcatccccagcgcacaggctgaaggaaatacatcgcacgggagagtcagaagcctcctgttttcccctcggacaagctgatttccttttgaaagcgggggacggtgaagaaaatgaagccaagcccgcaatttttgttagctgcttttctgtccttgattctgcagacggggatttgctatgggataaaatggatagctctgtccaagactccttcggctttggccctgaatcaaacccagcactgcaagcagctagaaggcctggtggtttctcaagtgcaattgtgccgcagcaacctggagctgatgcagaccatcatccaggcagcacgggaagtcataaagacctgccgtaaaactttctcagacatgcggtggaactgctcttccattgagctggctcctaactacctgctggacttagagagaggtacaagggagtcagcatttgtgtatgccctttctgctgctgccatcagccataccattgccagagcctgcaccaccggggacctccctggctgttcctgtggtcccatcccaggtgagacacctggacctgggtatcgatggggaggatgtgcagacaacctcaactatggtcttatcatggggtccaaattttcagatgctcccatgaagatgaaaaaatcaggatcacaagccaataaactgatgcatctgcacaacagtgaagtagggagacaggtcttgaaagcctctcttgaaatgaaatgtaagtgccatggagtttctgggtcgtgctctatcaagacctgttggaaaggccttcaagagctgcgagacattgcactggacctcaaaaacaagtatttgtcagctaccaaggtcgttcatcggcccatgggcacacgcaaatacctcgtgccaaaggatattgatatcaggccggttaaggagacagagctgatttacctgcagagctcgcctgatttctgcatgaagaatgagaaagtggggtcacacgggacccaggacaggcaatgcaacaagacctccaatgggagcgacagctgtgacctgatgtgctgcggcagaggctacaacccctacatggacaaagtggtggagaggtgccactgcaaataccactggtgctgctacgtcacctgtaaaaagtgtgagaggactgtcgagagatacgtctgcaaatgaaaacacctcgctctgaggctggatgttcagagaacagaaatgccttcatcttgtacagacatagaacttg

SEQ ID NO: 17 - DNA, 1927 bp, Homo sapiens
taacccgccgcctccgctctccccggctgcaggcggcgtgcaggaccagcggcggccgtgcaggcggaggacttcggcgcggctcctcctgggtgtgaccccgggcgcgcccgccgcgcgacgatgagggcgcggccgcaggtctgcgaggcgctgctcttcgccctggcgctccagaccggcgtgtgctatggcatcaagtggctggcgctgtccaagacaccatcggccctggcactgaaccagacgcaacactgcaagcagctggagggtctggtgtctgcacaggtgcagctgtgccgcagcaacctggagctcatgcacacggtggtgcacgccgcccgcgaggtcatgaaggcctgtcgccgggcctttgccgacatgcgctggaactgctcctccattgagctcgcccccaactatttgcttgacctggagagagggacccgggagtcggccttcgtgtatgcgctgtcggccgccgccatcagccacgccatcgcccgggcctgcacctccggcgacctgcccggctgctcctgcggccccgtcccaggtgagccacccgggcccgggaaccgctggggaggatgtgcggacaacctcagctacgggctcctcatgggggccaagttttccgatgctcctatgaaggtgaaaaaaacaggatcccaagccaataaactgatgcgtctacacaacagtgaagtggggagacaggctctgcgcgcctctctggaaatgaagtgtaagtgccatggggtgtctggctcctgctccatccgcacctgctggaaggggctgcaggagctgcaggatgtggctgctgacctcaagacccgatacctgtcggccaccaaggtagtgcaccgacccatgggcacccgcaagcacctggtgcccaaggacctggatatccggcctgtgaaggactcggaactcgtctatctgcagagctcacctgacttctgcatgaagaatgagaaggtgggctcccacgggacacaagacaggcagtgcaacaagacatccaacggaagcgacagctgcgaccttatgtgctgcgggcgtggctacaacccctacacagaccgcgtggtcgagcggtgccactgtaagtaccactggtgctgctacgtcacctgccgcaggtgtgagcgtaccgtggagcgctatgtctgcaagtgaggccctgccctccgccccacgcaggagcgaggactctgctcaaggaccctcagcaactggggccaggggcctggagacactccatggagctctgcttgtgaattccagatgccaggcatgggaggcggcttgtgctttgccttcacttggaagccaccaggaacagaaggtctggccaccctggaaggagggcaggacatcaaaggaaaccgacaagattaaaaataacttggcagcctgaggctctggagtgcccacaggctggtgtaaggagcggggcttgggatcggtgagactgatacagacttgacctttcagggccacagagaccagcctccgggaaggggtctgcccgccttcttcagaatgttctgcgggaccccctggcccaccctggggtctgagcctgctgggcccaccacatggaatcactagcttgggttgtaaatgttttcttttgttttttgctttttcttcctttgggatgtggaagctacagaaatatttataaaacatagctttttctttggggtggcacttctcaattcctctttatatattttatatatataaatatatatgtatatatataatgatctctattttaaaactagctttttaagcagctgtatgaaataaatgctgagtgagccccagcccgcccctgcagttcccggcctcgtcaagtgaactcggcagaccctggggctggcagagggagctctccagtttccaggca

(19) United States
(12) Patent Application Publication
(10) Pub. No.: US 2011/0052551 A1
(43) Pub. Date: Mar. 3, 2011

(54) CARDIAC MYOCYTE MORPHOGENIC COMPOSITIONS AND METHODS OF USE THEREFOR

(76) Inventors: Michael Patrick Flaherty, Louisville, KY (US); Buddhadeb Dawn, Missiion Hills, KS (US); Roberto Bolli, Louisville, KY (US)

(21) Appl. No.: 12/919,648

(22) PCT Filed: Feb. 26, 2009
(86) PCT No.: PCT/US2009/035272

§ 371 Date: Nov. 8, 2010

Related U.S. Application Data
(60) Provisional application No. 61/031,481, filed on Feb. 26, 2008.

(51) Int. Cl. A61K 35/28; C12N 5/071; C12N 5/10; C12M 3/00; A61P 9/10; A61P 9/00

(52) U.S. Cl. 424/93.7; 435/377; 435/325; 435/366; 435/370; 435/289.1

(57) ABSTRACT

Disclosed are methods for inducing cardiomyogenic differentiation in cells that are competent for differentiation along the cardiomyogenic lineage such as certain unfractionated bone marrow mononuclear cells (BMMNCs). In some embodiments, the methods include contacting a plurality of unfractionated, density gradient-separated BMMNCs with a cardiomyocyte differentiation-inducing amount of a Wnt11 gene product for a time and under conditions sufficient to induce cardiomyocyte differentiation in at least a subset of the BMMNCs. Also provided are methods for treating an injury to cardiac tissue in a subject using cells that have been induced to differentiate along the cardiomyogenic lineage, recombinant host cells comprising an expression vector that encodes a Wnt11 polypeptide or a functional fragment thereof, and systems for inducing cardiomyogenic differentiation in a cultured cell.

### CROSS REFERENCE TO RELATED APPLICATIONS

This application is based on and claims priority to U.S. Provisional Application Ser. No. 61/031,481, filed Feb. 26, 2008, the disclosure of which is incorporated herein by reference in its entirety.

### TECHNICAL FIELD

The presently disclosed subject matter relates to compositions that are capable of inducing differentiation along the cardiomyogenic lineage. Also provided are methods and systems for inducing cardiomyogenic differentiation in cells.

### BACKGROUND

The bone marrow (BM) is an easily accessible source of autologous adult stem and progenitor cells, and mounting evidence supports the potential utility of BM-derived cells (BMCs) for somatic tissue repair (Herzog et al., 2003; Dawn & Bolli, 2005; Abdel-Latif et al., 2007). The mononuclear fraction of BMCs (unfractionated BMMNCs) represents a phenotypically heterogeneous, mixed population of committed and uncommitted cells. Preclinical studies using an enriched sub-population of BMMNCs have revealed the ability of BMCs to restore structure and function in dead myocardium (Orlic et al., 2001). The cardiac reparative potential of unfractionated BMMNCs has subsequently been validated by the results of clinical trials (Strauer et al., 2002; Schachinger et al., 2006; Abdel-Latif et al., 2007). Because of this, and because of the ease of preparation, BMMNCs are currently the most widely used and readily available cells for cardiac repair in humans. However, since tracking cardiomyocytic differentiation is not clinically feasible, the mechanism of the salubrious effects of BMMNCs is controversial, and it is not known whether unfractionated BMMNCs acquire a cardiac phenotype once implanted in humans. Although the cardiomyogenic potential of various subsets of BMCs and other adult primitive cells has been investigated, the ability of unfractionated BMMNCs to undergo cardiac commitment remains unclear.

To elucidate this issue, the effects of Wnt3a and Wnt11 polypeptides on cardiac lineage commitment of unfractionated BMMNCs was investigated. The impact of these polypeptides on BMMNC proliferation, expression of markers of pluripotency, and reciprocal regulation of signaling was also investigated. The results presented herein indicated that in unfractionated BMMNCs, Wnt3a promoted hematopoietic commitment and sternness, while Wnt11 induced cardiac lineage commitment in a PKC-dependent manner and contributed to improvement in cardiac function following intramyocardial implantation.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides methods for inducing cardiomyogenic differentiation in isolated bone marrow mononuclear cells (BMMNCs). In some embodiments, the methods comprise contacting a plurality of BMMNCs with a cardiomyocyte differentiation-inducing amount of a Wnt11 gene product or a functional fragment thereof for a time and under conditions sufficient to induce cardiomyocyte differentiation in at least a subset of the BMMNCs. In some embodiments, the Wnt11 gene product comprises a Wnt11 polypeptide or a functional fragment thereof. In some embodiments, the Wnt11 polypeptide or the functional fragment thereof is present in a conditioned medium recovered from or produced by a cell culture in which at least one cell that conditioned the medium secreted the Wnt11 polypeptide or the functional fragment thereof. In some embodiments, the Wnt11 gene product is a human Wnt11 gene product.

In some embodiments, the contacting with the Wnt11 gene product is accomplished without contacting the plurality of BMMNCs with any cells or cell types other than BMMNCs. In some embodiments, the contacting occurs as the Wnt11 gene product traverses a membrane or other barrier that physically separates the BMMNCs from a source of the Wnt11 gene product or the functional fragment thereof.

In some embodiments of the presently disclosed methods, the source of the Wnt11 gene product is a cell in culture that expresses the Wnt11 gene product naturally or has been manipulated to express an endogenous or a recombinant Wnt11 coding sequence, and further wherein the endogenous or recombinant Wnt11 coding sequence encodes a Wnt11 gene product or a functional fragment thereof. In some embodiments, the time and conditions sufficient to induce cardiomyocyte differentiation in at least a subset of the

BMMNCs induces expression of at least one gene including, but not limited to cardiac troponin T (cTnT), cardiac myosin heavy chain (cMyHC)], and connexin 43.

The presently disclosed subject matter also provides methods for treating an injury to cardiac tissue in a subject, the method comprising administering to the subject a composition comprising a plurality of Wnt11-induced bone marrow mononuclear cells (BMMNCs) in a pharmaceutically acceptable carrier, in an amount and via a route sufficient to allow at least a fraction of the plurality of Wnt11-induced BMMNCs to contact the cardiac tissue, whereby the injury is treated. In some embodiments, the injury includes, but is not limited to an ischemic injury and a myocardial infarction. In some embodiments, the subject is a mammal. In some embodiments, the method further comprises differentiating the Wnt11-induced BMMNCs to produce a plurality of cardiomyocytes or precursor cells thereof. In some embodiments, the cardiomyocytes or precursor cells thereof express at least one gene including, but not limited to cardiac troponin T (cTnT), cardiac myosin heavy chain (cMyHC)], and connexin 43.

The presently disclosed subject matter also provides recombinant host cells comprising an expression vector that encodes a Wnt11 polypeptide or a functional fragment thereof. In some embodiments, the Wnt11 polypeptide or a functional fragment thereof is secreted from the recombinant host cell. In some embodiments, the recombinant host cell is an isolated or immortalized human cell, optionally a human embryonic kidney-293 (HEK-293) cell. In some embodiments, the expression vector comprises a nucleic acid sequence encoding a Wnt11 polypeptide or a functional fragment thereof operably linked to a promoter, optionally a constitutive promoter, which is active in the recombinant host cell. In some embodiments, the Wnt11 polypeptide comprises amino acids 1-354 of GENBANK® Accession No. P51891 (quail Wnt11), or a functional fragment thereof, which is at least 95% identical at the amino acid level to amino acids 1-354 of GENBANK® Accession No. P51891, optionally over the full 354 amino acid length of GENBANK® Accession No. P51891. In some embodiments, the Wnt11 polypeptide comprises amino acids 1-354 of GENBANK® Accession No. NP_{—}004617 (human Wnt11), or a functional fragment thereof, which is at least 95% identical at the amino acid level to amino acids 1-354 of GENBANK® Accession No. NP_{—}004617, optionally over the full 354 amino acid length of GENBANK® Accession No. NP_{—}004617.

The presently disclosed subject matter also provides systems for inducing cardiomyogenic differentiation in a cultured cell. In some embodiments, the systems comprise (a) a source of a Wnt11 polypeptide; and (b) a growth area in which the cell is cultured; and optionally (c) a barrier that physically separates the source of the Wnt11 polypeptide from the cultured cell that is permeable to the Wnt11 polypeptide, thereby allowing the Wnt11 polypeptide provided by the source to contact the cultured cell. In some embodiments, the source of the Wnt11 polypeptide comprises a second cell that expresses a secretable Wnt11 polypeptide, and the barrier prevents physical contact between the second cell that expresses the secretable Wnt11 polypeptide and the cultured cell in which cardiomyogenic differentiation is to be induced. In some embodiments, the second cell is a recombinant cell that comprises an expression vector encoding the secretable Wnt11 polypeptide. In some embodiments, the Wnt11 polypeptide comprises an amino acid sequence including, but not limited to (a) amino acids 1-354 of GENBANK® Accession No. P51891; (b) amino acids 1-354 of GENBANK® Accession No. NP_{—}004617; (c) a functional fragment of (a) or (b); and/or (d) an amino acid sequence at least 95% identical to either (a) or (b), wherein the Wnt11 polypeptide induces cardiomyogenic differentiation in the cultured cell.

It is an object of the presently disclosed subject matter to provide compositions that are capable of inducing differentiation along the cardiomyogenic lineage.

An object of the presently disclosed subject matter having been stated hereinabove, and which is achieved in whole or in part by the presently disclosed subject matter, other objects will become evident as the description proceeds when taken in connection with the accompanying drawings and Examples as best described hereinbelow.

### BRIEF DESCRIPTION OF THE DRAWINGS

FIGS. 1A-1C depict Wnt11 protein and mRNA expression by Wnt11/293 stable transfectants.

FIG. 1A depicts a Western blot wherein each lane contains proteins isolated from either cellular extracts or media from individual Wnt11/293 cell cultures (Wnt11/293-1 or Wnt11/293-2 clones) or pCDNA3-293 cell cultures (empty vector). FIG. 1B is a bar graph depicting densitometric analysis of Wnt11 protein levels normalized to the loading control (GAPDH) expressed as a percent of control (empty vector). FIG. 1C is a bar graph depicting qRT-PCR analysis of Wnt11 mRNA expression in Wnt11/293 cells, expressed as transcript abundance relative to internal control (18S rRNA). Data are mean±standard error of the mean (SEM). *P<0.001 vs. empty vector (FIG. 1B) or Wnt11/293-1 (FIG. 1C).

FIGS. 2A-2E depict expression of markers of pluripotency and proliferation rates in cultured unfractionated BMMNCs.

FIGS. 2A and 2B are bar graphs depicting quantitative reverse transcription-polymerase chain reaction (qRT-PCR) analysis of Oct-4 (FIG. 2A) and Nanog (FIG. 2B) mRNA expression in freshly isolated unfractionated bone marrow mononuclear cells (BMMNCs) at baseline and in BMMNCs cultured for 21 days with empty vector (control), Wnt3a, or Wnt11-conditioned medium (Wnt11-CM). mRNA levels in unfractionated BMMNCs after 21 days of culture were normalized to 18S rRNA (internal control) and expressed as a “fold difference” relative to baseline (calibrator). Assays were performed in triplicate. Data are mean±SEM from three independent experiments.

FIGS. 2C and 2D depict representative merged confocal images of BMMNCs after 21 days of culture in Wnt3a shows nuclear localization of Oct-4.

FIG. 2E is a graph depicting -fold increase in cell number versus baseline (y-axis) versus days of proliferation (x axis). Cultures were initiated with a total of 10⁴ cells (0 days on the graph) in each group. Scale bar=25 μm. Close circle: control; Square: Wnt3a; Open circle: Wnt11-CM; *: P<0.01 vs. control and Wnt11-CM.

FIGS. 3A-3G depict morphologic and phenotypic features of unfractionated BMMNCs cultured for 21 days in the presence or absence of Wnt3a.

FIG. 3A is a representative transmission image showing significant differentiation and morphological heterogeneity in BMMNCs after 21 days in control medium: large stromal network (enclosed by black bracket) containing fibroblastic (black arrows) and epithelial-like cells with large nuclei (white arrow); endothelial-like cells (white arrowhead) are also present. FIG. 3B is a transmission image of BMMNCs cultured in the presence of 150 ng/ml of recombinant Wnt3a protein showing cells at various stages of hematopoiesis: myeloblastic cells with increased nuclear:cytoplasmic ratio (white arrows), myelocytic/metamyelocytic cells with eccentric nuclei (black arrowheads), fibroblastic (long black arrow), and endothelial-like cells (white arrowhead) are also present, though in much lower numbers.

FIGS. 3C-3F are confocal microscopic images of BMMNCs cultured in empty vector (control) medium (FIGS. 3C and 3D) showing absence of fluorescence denoting lack of expression of the pan-hematopoietic marker CD45. BMMNCs cultured in presence of Wnt3a (FIGS. 3E and 3F) after 21 days of culture showed marked positivity for CD45 (FIG. 3F; dark fluorescence). All nuclei are stained with DAPI (light fluorescence).

FIG. 3G is a bar graph depicting quantitative assessment of CD45 expression in BMMNCs after 21 days of culture in control medium, in Wnt11-CM, and in the presence of Wnt3a. Cells positive for CD45 are expressed as a percent of total number of cells in culture. Data are mean±SEM of three independent experiments. Scale bar=25 μm for FIGS. 3A and 3B and 30 μm for FIGS. 3C-3F).

FIGS. 4A-4Q depict photomicrographic images showing morphological and phenotypic features of unfractionated BMMNCs cultured for 21 days in Wnt11-CM. Scale bar=25 μm.

FIGS. 4A and 4D are representative transmission images showing cells at various stages of differentiation with morphologies characteristic of differentiating cardiomyocytes: enlarged elliptical shape (white arrows), some with short cytoplasmic processes (white arrowhead); rod-shaped (asterisk); or elongated (black arrowhead). FIGS. 4B, 4C, 4E, and 4F are confocal microscopic images of the same BMMNCs as depicted in FIGS. 4A and 4D stained with DAPI (FIGS. 4B and 4E) and showing expression of the cardiac-specific contractile proteins cardiac myosin heavy chain (cMyHC) and cardiac troponin T (cTnT; FIGS. 4C and 4F, respectively; fluorescence), indicating cardiomyogenic differentiation.

FIGS. 4G-4I are confocal images of BMMNCs after 21 days of culture in Wnt11-CM stained with DAPI (FIG. 4G) and demonstrating expression of connexin-43 (FIGS. 4H and 4I; fluorescence).

FIGS. 4J-4M show co-localization (FIG. 4M) of connexin-43 (FIGS. 4K and 4M) and cTnT (FIGS. 4L and 4M). BMMNCs cultured under the same conditions underwent co-immunostaining and revealed cTnT expression (FIGS. 4P and 4Q) and the absence of the skeletal muscle-specific protein myogenin (FIG. 4O). All nuclei are stained with DAPI.

FIGS. 5A-5Q show comparative effects of Wnt3a and Wnt11 on cardiomyogenic differentiation of unfractionated BMMNCs. BMMNCs were cultured for 21 days either in the presence of recombinant Wnt3a protein (Wnt3a), in co-culture with Wnt11 secreting cells (Wnt11-conditioned medium, Wnt11-CM), or in co-culture with pcDNA3/293 empty vector (control). All data are mean±SEM of three independent experiments. Scale bar=40 μm.

FIGS. 5A-5L depict representative confocal microscopic images of cells after immunocytochemical staining for cardiac troponin T (cTnT) and cardiac myosin heavy chain (cMyHC). Expression of cTnT (FIGS. 5A and 5B) and cMyHC (FIGS. 5C and 5D) were detected only in BMMNCs co-cultured in Wnt11-CM, indicating cardiomyogenic differentiation. Expression of cardiac-specific contractile proteins was not observed in cells cultured with Wnt3a (FIGS. 5E-5H) or control media (FIGS. 5I-5L).

FIGS. 5M-5P show that expression of cTnT and cMyHC were not detected in Wnt11-expressing 293 cells (Wnt11/293-2). All nuclei are stained with DAPI.

FIG. 5Q is a bar graph showing the number of cMyHC and cTnT-expressing cells after 21 days of culture of BMMNCs with Wnt11-CM, empty vector, or Wnt3a. Cells expressing cTnT and cMyHC were not observed after culture with Wnt3a or empty vector control. Cells positive for cMyHC and cTnT are expressed as a percent of total cells in culture.

FIGS. 6A-6F are a series of bar graphs showing Wnt11-induced activation of the cardiac gene program in unfractionated BMMNCs. Cells were cultured for 21 days either in the presence of 150 ng/ml of recombinant Wnt3a protein or in co-culture with Wnt11 secreting cells (Wnt11/293-2) or pcDNA3/293 empty vector (control) cells. Quantitative assessment of expression of cardiac-specific transcription factors Nk×2.5 (FIG. 6A) and GATA-4 (FIG. 6B), atrial natriuretic peptide (ANP; FIG. 6C), and contractile proteins cTnT (FIG. 6D), with α-MyHC (FIG. 6E), and β-MyHC (FIG. 6F) isoforms was performed by qRT-PCR at days 0, 3, 7, and 21. FIGS. 6A-6F show that no expression of cardiac-specific markers was detected prior to induction of BMMNCs (day 0). No expression of cardiac-specific markers was observed in control and Wnt3a-treated BMMNCs during 21 days of culture. mRNA levels in unfractionated BMMNCs cultured in Wnt11-CM were normalized to 18S rRNA (internal control) and expressed as -fold differences, relative to first detectable mRNA expression after induction (day 3). All samples were run in triplicate. Data are mean±SEM of three independent experiments. *P<0.001 vs. respective levels on day 3; #P<0.001 vs. respective levels on day 7.

FIGS. 7A-7C are a series of bar graphs showing Wnt and β-catenin expression in unfractionated BMMNCs. All samples were run in triplicate. Data are mean±SEM of three independent experiments.

FIGS. 7A and 7B depict the results of quantitative assessments of Wnt3a (FIG. 7A) and β-catenin (FIG. 7B) expression by qRT-PCR in freshly isolated BMMNCs (baseline), and in BMMNCs cultured with pCDNA3/293 cells (control), in presence of 150 ng/ml of recombinant Wnt3a protein (Wnt3a), or in presence of Wnt11/293-2 cells (Wnt11-CM) for 21 days. mRNA levels from unfractionated BMMNCs following 21 days of culture are normalized to 18S rRNA (internal control) and expressed as -fold difference relative to baseline (calibrator). No expression of Wnt11 was observed in unfractionated BMMNCs or in empty vector (control) cells. Solid line: baseline; gray box: control; stippled box: Wnt3a; hatched box: Wnt11-CM, *P<0.001 vs. baseline; #P<0.001 vs. control; §P<0.001 vs. Wnt11-CM.

FIG. 7C depicts quantitative assessment of cTnI was performed using ELISA in BMMNCs cultured for 21 days with no addition (control), with the canonical Wnt inhibitor Dickkopf-1 (Dkk-1), or in Wnt11-CM without (Wnt11-CM) or with Dkk-1 (Wnt11-CM+Dkk-1). Protein levels are expressed as percent of control. *P<0.001 vs. control; §P<0.001 vs. Dkk-1; ‡P<0.001 vs. Wnt11-CM+Dkk-1.

FIGS. 8A and 8B depict the role of PKC and JNK signaling in Wnt11-mediated cardiac gene expression.

FIG. 8A is a series of immunoblots and a bar graph showing expression and expression levels of JNK, phospho-JNK (p-JNK), and GAPDH levels in cultured BMMNCs. In FIG. 8A, BMMNCs were cultured with (Wnt11-CM) or without soluble Wnt11 protein (control), and with Wnt11-CM in the presence of the PKC inhibitor bisindolylmaleimide I (BIM; Wnt11+BIM). The top panel depicts immunoblots showing JNK, phospho-JNK (p-JNK), and GAPDH levels in cultured BMMNCs. The bottom panel is a bar graph of densitometric analyses of JNK and p-JNK signals normalized to GAPDH and expressed as percent of control. Data are mean±SEM. Gray box: control; stippled box: Wnt11-CM; hatched box: Wnt11-CM+BIM; *P<0.001 vs. control; #P<0.001 vs. Wnt11-CM+BIM.

FIG. 8B is a series of bar graphs depicting expression of various markers in BMMNCs cultured for 3 days in Wnt11-CM alone (control) or in Wnt11-CM in the presence of either BIM or the JNK inhibitor SP600125. By qRT-PCR, no expression of cardiac-specific transcription factors (Nk×2.5 and GATA-4), atrial natriuretic peptide (ANP), and contractile proteins (cTnT, α-, and β-MyHC isoforms) could be detected in BMMNCs treated with Wnt11-CM+BIM. In BMMNCs treated with Wnt11-CM+SP600125, the expression of ANP was relatively unaffected, that of Nk×2.5 and GATA-4 was significantly reduced, and cardiac-specific structural proteins were undetectable. mRNA levels are normalized to 18S rRNA (internal control) and expressed as fold-differences, relative to levels observed in Wnt11-CM-treated (control) BMMNCs. All samples were run in triplicate. All mRNA levels are expressed as -fold change vs. their expression levels on day 3 (indicated as 1 on the x-axis), which are the control levels. Data are mean±SEM of three independent experiments.

FIGS. 9A-9C schematically depict exemplary experimental designs for each protocol described herein with unfractionated bone marrow mononuclear cells (BMMNCs).

FIG. 9A schematically depicts designs using density-gradient centrifugation in which the mononuclear cell fraction was purified from freshly isolated murine BM. These BMMNCs were cultured for 10 days in complete medium to allow for maximal attachment of all sub-populations. Next, inserts containing pcDNA/293 (control) and Wnt11/293-2 (Wnt11-CM) cells were placed atop (in the upper chamber) the attached BMMNCs (in the lower chamber) and cultured for 21 days. In addition, attached BMMNCs were cultured separately in the presence of 150 ng/ml of soluble recombinant Wnt3a protein for 21 days and analyzed in the same manner. Respective cultures were analyzed for (a) proliferation; (b) cellular morphology and differentiation; and (c) mRNA expression. Freshly isolated BMMNCs were analyzed separately for baseline mRNA expression.

FIG. 9B schematically depicts an experimental design for analyzing mRNA and cardiac marker expression on purified and induced BMMNCs. On the left quarter of FIG. 9B, BMMNCs were purified as above and cultured for 3 hours in the presence of Wnt11-CM, with or without bisindolylmaleimide I (BIM, 1 μM) and analyzed by immunoblotting for the presence of phospho-JNK (p-JNK) and total JNK (JNK). The right three-quarters of FIG. 9B depict a design for induction and analysis of BMMNCs. BMMNCs were allowed to attach (as above) for 10 days, after which cells were cultured for 3 days in pcDNA/293 (vector/control) medium or in Wnt11-CM, with or without either BIM (1 μM) or the JNK inhibitor SP600125 (10 μM) and analyzed for mRNA expression of cardiac-specific markers.

FIG. 9C schematically depicts a design for assaying the expression of cardiac-specific troponin I (cTnI) by ELISA following culture in presence of Wnt11 with and without the Wnt inhibitor Dickkopf-1 (Dkk-1).

FIGS. 10A and 10B depict morphological features of BMMNCs prior to culture. Representative transmission microscopic images show: FIG. 10A shows BMMNCs 24 hours after plating (i.e., at day- 9), prior to culturing, with considerable morphologic variation. FIG. 10B depicts the boxed section of panel A at greater magnification to better demonstrate the morphological features of these primitive cell types: myeloblast-like cells with increased nucleocytoplasmic ratio (black arrows), large promyelocytic cells with eccentric nuclei (black arrowhead), smaller pre-granulocytic myelocytic/metamyelocytic cells with polar eccentric nuclei (white arrows) and granulocytes (white arrowheads). Scale bar=25 μm.

FIGS. 11A-11F depict confocal microscopic images showing the impact of Wnt3a on Oct-4 expression in cultured unfractionated BMMNCs. Representative images demonstrating Oct-4 expression in BMMNCs cultured in the absence (FIGS. 11A-11C) or presence (FIGS. 11D-11F) of Wnt3a (150 ng/ml) for 21 days. Oct-4 is identified in fluorescence in FIGS. 11A-11F. Nuclei are identified in fluorescence of DAPI in FIGS. 11B, 11C, 11E, and 11F. After 21 days, virtually no Oct-4-positive cells were observed in untreated BMMNCs (FIGS. 11A-11C). In contrast, a much greater number of Oct-4-positive cells were noted in BMMNCs cultured in presence of Wnt3a (FIGS. 11D-11F). Scale bar=25 μm.

FIG. 12 is a diagram depicting treatments and treatment groups of mice injected intramyocaridally with BMMNCs.

FIGS. 13A and 13B are representative confocal microscopic images demonstrating myocardial localization of murine Wnt11-treated bone marrow mononuclear cells (BMMNCs) from heterozygous-mutant green fluorescent protein (GFP) expressing transgenic mice (GFP−/+) 48 hours following their intramyocardial infusion into nude rats.

FIG. 13A is a merged image (×20; magnification) of longitudinal sections from nude rat myocardium showing interstitial accumulation of green (GFP-pos) BMMNCs exposed to Wnt11 for 3 days (preprogrammed) in vitro prior to their intramyocardialinjection.

FIG. 13B is a similar image to that in FIG. 13A, at a higher magnification (×60).

FIG. 14 is a bar graph of left ventricular ejection fractions at baseline (BSL) and at 35 days (35 d) for the mice of Groups I-III described in FIG. 12. *: P<0.001 vs. baseline; †: P<0.001 vs. Group I; #: P<0.001 vs. Group I and Group II.

FIG. 15 is a bar graph showing left ventricular end-diastolic volumes at baseline (BSL) and at 35 days (35 d) for the mice of Groups I-III described in FIG. 12. *: P<0.001 vs. baseline; †: P<0.001 vs. Group I; #: P<0.001 vs. Group I and Group II.

### BRIEF DESCRIPTION OF THE SEQUENCE LISTING

SEQ ID NOs: 1-17 relate to nucleic acid sequences of certain human gene products disclosed herein as set forth in the GENBANK® database. The sequences disclosed in the Sequence Listing are summarized in Table 1. The full disclosures of the GENBANK® Accession Nos. presented hereinbelow are incorporated herein by reference, including but not limited to all annotations and also corresponding amino acid sequence references presented therein.

TABLE 1
Sequences Disclosed in the Sequence Listing

| SEQ | GENBANK ® | |
|---|---|---|
| ID NO: | Acc. No. | Description |
| 1 | NM_000257 | β-MyHc gene product: myosin, |
| | | heavy chain 7, |
| | | cardiac muscle, beta; MYH7; |
| 2 | NM_002471 | α-MyHC gene product: myosin, |
| | | heavy chain 6, |
| | | cardiac muscle, alpha, cardiomyopathy, |
| | | hypertrophic 1; MYH6; |
| 3 | NM_000364 | cTnT: troponin T type 2 |
| | | (cardiac) (TNNT2), |
| | | transcript variant 1; |
| 4 | NM_001001430 | cTnT: troponin T type 2 |
| | | (cardiac) (TNNT2), |
| | | transcript variant 2; |
| 5 | NM_001001431 | cTnT: troponin T type 2 |
| | | (cardiac) (TNNT2), |
| | | transcript variant 3; |
| 6 | NM_001001432 | cTnT: troponin T type 2 |
| | | (cardiac) (TNNT2), |
| | | transcript variant 4; |
| 7 | NM_004387 | Nkx2.5: NK2 transcription factor |
| | | related, locus 5; Drosophila; NKX2-5; |
| 8 | NM_002052 | GATA-4: GATA binding |
| | | protein 4; GATA4; |
| 9 | NM_006172 | ANP: natriuretic peptide |
| | | precursor A; NPPA; |
| 10 | NM_002521 | ANP: natriuretic peptide |
| | | precursor B; NPPB; |
| 11 | NM_001904 | β-catenin: catenin; |
| | | cadherin-associated protein; |
| | NP_001895 | beta 1, 88 kDa; CTNNB1; |
| 12 | NM_002701 | Oct-4: POU Class 5 Homeobox |
| | | 1; POU5F1; |
| | | Transcript Variant 1; |
| 13 | NM_203289 | Oct-4: POU Class 5 Homeobox |
| | | 1; POU5F1; |
| | | Transcript Variant 2; |
| 14 | NM_024865 | Nanog: Nanog Homeobox; NANOG; |
| 15 | NR_003286 | Homo sapiens 18 S ribosomal RNA |
| | | (LOC100008588); |
| 16 | X97549 | Wnt11 (quail); |
| 17 | NM_004626 | wingless-type MMTV |
| | | integration site family, |
| | | member 11 precursor; Wnt11 (human). |

### DETAILED DESCRIPTION

The present subject matter will be now be described more fully hereinafter with reference to the accompanying Examples, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### I. General Considerations

Disclosed herein are compositions and methods for isolating and differentiating unfractionated bone marrow mononuclear cells (BMMNCs) along the cardiomyogenic lineage. Unfractionated BMMNCs were isolated from adult mice via FICOLL-PAQUE™ density-gradient centrifugation and cultured in presence of Wnt3a or Wnt11. Wnt11 was not expressed in control BMMNCs, while the expression of markers of pluripotency (Oct-4 and Nanog), as well as that of Wnt3a and β-catenin, decreased progressively during culture. Exposure to Wnt3a rescued β-catenin expression and markedly increased expression of Oct-4 and Nanog, concomitant with increased cell proliferation and CD45 expression. In contrast, exposure to ectopically expressed non-canonical Wnt11 markedly decreased the expression of Oct-4 and Nanog and induced mRNA expression of cardiac-specific genes (Nk×2.5, GATA-4, atrial natriuretic peptide, α-myosin heavy chain (α-MyHC), β-myosin heavy chain (β-MyHC), and troponin T (cTnT)) by day 3 with subsequent progression to a pattern characteristic of the cardiac fetal gene program. After 21 days, 27.6±0.6% and 29.6±1.4% of BMMNCs expressed the cardiac-specific antigens cMyHC and cTnT, respectively, indicating cardiomyogenic lineage commitment. Wnt11-induced cardiac-specific expression was completely abolished by the PKC inhibitor bisindolylmaleimide I, partially abolished by the JNK inhibitor SP600125, and attenuated by the Wnt inhibitor Dickkopf-1.

BMMNCs that underwent Wnt11-induced cardiomyocytic lineage commitment, referred to herein as “cardiomyogenic preprogramming”, were subsequently tested in vivo to validate the concept of Wnt11-mediated enhancement of BMMNC myocardial reparative potential. Thus, also disclosed are methods for employing the isolated unfractionated bone marrow mononuclear cells (BMMNCs) that have been differentiated along the cardiomyogenic lineage in a murine experimental infarction model by intramyocardial implantation.

Thus, in adult density-gradient separated BMMNCs, canonical Wnt3a promoted “sternness”, proliferation, and hematopoietic commitment, while non-canonical signaling via Wnt11 induced robust cardiomyogenic differentiation in a PKC- and JNK-dependent manner.

### II. Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” mean “one or more” when used in this application, including the claims. Thus, the phrase “a stem cell” refers to one or more stem cells, unless the context clearly indicates otherwise.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Ayes (birds), and Mammalia (mammals)), and all Orders and Families encompassed therein.

Similarly, all genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes listed in Tables 1 and 2, which disclose GENBANK® Accession Nos. for the murine and human nucleic acid sequences, respectively, are intended to encompass homologous genes and gene products from other animals including, but not limited to other mammals, fish, amphibians, reptiles, and birds.

The presently disclosed subject matter is particularly useful for warm-blooded vertebrates. Thus, the presently disclosed subject matter concerns mammals and birds. More particularly provided is the isolation, manipulation, and use of subpopulations of BMMNCs from mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the isolation, manipulation, and use of a subpopulation of BMMNCs from livestock, including but not limited to domesticated swine (pigs and hogs), ruminants, horses, poultry, and the like.

The term “about”, as used herein when referring to a measurable value such as an amount of weight, time, dose, etc., is meant to encompass variations of in some embodiments ±20%, in some embodiments ±10%, in some embodiments ±5%, in some embodiments ±1%, and in some embodiments ±0.1% from the specified amount, as such variations are appropriate to perform the disclosed methods.

The term “isolated”, as used in the context of a cell (including, for example, a BMMNC), indicates that the cell exists apart from its native environment. An isolated cell can also exist in a purified form or can exist in a non-native environment.

As used herein, a cell exists in a “purified form” when it has been isolated away from all other cells that exist in its native environment, but also when the proportion of that cell in a mixture of cells is greater than would be found in its native environment. Stated another way, a cell is considered to be in “purified form” when the population of cells in question represents an enriched population of the cell of interest, even if other cells and cell types are also present in the enriched population. A cell can be considered in purified form when it comprises in some embodiments at least about 10% of a mixed population of cells, in some embodiments at least about 20% of a mixed population of cells, in some embodiments at least about 25% of a mixed population of cells, in some embodiments at least about 30% of a mixed population of cells, in some embodiments at least about 40% of a mixed population of cells, in some embodiments at least about 50% of a mixed population of cells, in some embodiments at least about 60% of a mixed population of cells, in some embodiments at least about 70% of a mixed population of cells, in some embodiments at least about 75% of a mixed population of cells, in some embodiments at least about 80% of a mixed population of cells, in some embodiments at least about 90% of a mixed population of cells, in some embodiments at least about 95% of a mixed population of cells, and in some embodiments about 100% of a mixed population of cells, with the proviso that the cell comprises a greater percentage of the total cell population in the “purified” population that it did in the population prior to the purification. In this respect, the terms “purified” and “enriched” can be considered synonymous.

The term “isolated”, as used in the context of a nucleic acid or polypeptide (including, for example, a peptide), indicates that the nucleic acid or polypeptide exists apart from its native environment. An isolated nucleic acid or polypeptide can exist in a purified form or can exist in a non-native environment.

The terms “nucleic acid molecule” and “nucleic acid” refer to deoxyribonucleotides, ribonucleotides, and polymers thereof, in single-stranded or double-stranded form. Unless specifically limited, the term encompasses nucleic acids containing known analogues of natural nucleotides that have similar properties as the reference natural nucleic acid. The terms “nucleic acid molecule” and “nucleic acid” can also be used in place of “gene”, “cDNA”, and “mRNA”. Nucleic acids can be synthesized, or can be derived from any biological source, including any organism.

Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Ohtsuka et al., 1985; Batzer et al., 1991; Rossolini et al., 1994). The terms “nucleic acid” or “nucleic acid sequence” can also be used interchangeably with gene, open reading frame (ORF), cDNA, and mRNA encoded by a gene.

### III. Methods for Inducing Cardiomyogenic Differentiation

The presently disclosed subject matter provides methods for inducing cardiomyogenic differentiation in cells that are competent for differentiating into cardiomyocytes and/or their precursors. In some embodiments, the methods comprise inducing cardiomyogenic differentiation in unfractionated, density gradient-separated bone marrow mononuclear cells (BMMNCs). Methods for separating such BMMNCs are known in the art, and are also disclosed herein.

In some embodiments, the methods comprise contacting a plurality of BMMNCs with a cardiomyogenic differentiation-inducing amount of a Wnt11 gene product for a time and under conditions sufficient to induce cardiomyogenic differentiation in at least a subset of the BMMNCs. As used herein, the phrase “Wnt11 gene product” refers to a Wnt11 polypeptide or a functional fragment thereof, or a nucleic acid sequence encoding the same. In some embodiments, the Wnt11 gene product is a polypeptide encoded by a Wnt11 genetic locus (e.g., a human Wnt11 genetic locus), or a functional fragment thereof.

As used herein, the phrase “functional fragment thereof” refers to a polypeptide that has an amino acid sequence that is a subsequence of a naturally occurring Wnt11 polypeptide, or of a variant thereof as defined hereinbelow, wherein the functional fragment is capable of inducing at least a fraction of the cardiomyogenic differentiation in that fraction of BMMNCs in which a full length Wnt11 gene product is capable of inducing cardiomyogenic differentiation. A functional fragment induces in some embodiments at least 5%, in some embodiments at least 10%, in some embodiments at least 15%, in some embodiments at least 20%, in some embodiments at least 25%, in some embodiments at least 30%, in some embodiments at least 35%, in some embodiments at least 40%, in some embodiments at least 45%, in some embodiments at least 50%, in some embodiments at least 55%, in some embodiments at least 60%, in some embodiments at least 65%, in some embodiments at least 70%, in some embodiments at least 75%, in some embodiments at least 80%, in some embodiments at least 85%, in some embodiments at least 90%, in some embodiments at least 95%, or in some embodiments at least 99% of the degree of cardiomyogenic differentiation in that fraction of BMMNCs in which a full length Wnt11 is capable of inducing cardiomyogenic differentiation.

It is also understood that the phrase “Wnt11 gene product” includes variants of a naturally occurring Wnt 11 gene product, wherein the variants comprise one or more amino acid or nucleotide deletions, substitutions, or additions when compared to a naturally occurring Wnt 11 gene product, with the proviso that the variant retains at least some percentage (e.g., at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% or more of a particular biological activity of a naturally occurring Wnt11 gene product upon which it is based. For example, a variant of a Wnt11 gene product can include one or more conservative amino acid substitutions relative to a naturally occurring Wnt11 gene product while retaining at least some percentage of the cardiomyogenic differentiation activity of the naturally occurring gene product.

The term “conservative substitution”, and grammatical variants thereof, refers in some embodiments to a polypeptide (e.g., a Wnt11 gene product) comprising an amino acid residue sequence substantially identical to an amino acid residue of a reference sequence in which one or more amino acid residues have been conservatively substituted with a functionally similar residue and which displays at least a fraction of the Wnt11 biological activity as described herein. Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

Polypeptides of the presently disclosed subject matter also include peptides comprising one or more additions and/or deletions or residues relative to the sequence of a polypeptide whose sequence is disclosed herein, so long as at least a fraction of the Wnt11 biological activity disclosed herein is maintained. The term “fragment” refers to a polypeptide comprising an amino acid residue sequence shorter than that of a polypeptide disclosed herein.

When variant Wnt11 gene products are generated, the amino acid sequence of the variant Wnt11 gene product can be 80%, 85%, 90%, 91%, 92%, 93%, 94%, 95%, 96%, 97%, 98%, or 99% or more identical to that amino acid sequence of the naturally occurring Wnt11 gene product upon which the variant is based (e.g., a naturally occurring mammalian Wnt11 polypeptide or a functional fragment thereof). Algorithms that can be employed for determining percent identity include the BLAST algorithms available to the public through the website of the National Center for Biotechnology Information (NCBI). In some embodiments, the percent identity is over the full length of the amino acid sequence of a naturally occurring Wnt11 gene product. In some embodiments, the percent identity is over the full length of any overlap between the variant Wnt11 gene product and a naturally occurring Wnt11 gene product upon which the variant is based.

In some embodiments, the cardiomyogenic differentiation-inducing activity of a Wnt11 gene product is provided by a small molecule mimetic. The term “small molecule mimetic” as used herein refers to a ligand that mimics the biological activity of a reference Wnt11 gene product (e.g., a Wnt11 polypeptide or a functional fragment thereof), by substantially duplicating the targeting activity of the reference Wnt11 gene product, but it is not a polypeptide or a peptoid per se. In some embodiments, a small molecule mimetic is a peptide mimetic has a molecular weight of less than about 700 daltons.

A small molecule mimetic can be designed by: (a) identifying the pharmacophoric groups responsible for the biological activity of a Wnt11 gene product; (b) determining the spatial arrangements of the pharmacophoric groups in the active conformation of the Wnt11 gene product; and (c) selecting a pharmaceutically acceptable template upon which to mount the pharmacophoric groups in a manner that allows them to retain their spatial arrangement in the active conformation of the Wnt11 gene product. For identification of pharmacophoric groups responsible for biological activity (e.g., cardiomyogenic differentiation-inducing activity), mutant variants of the Wnt11 gene product can be prepared and assayed for differentiation-inducing activity. Alternatively or in addition, the three-dimensional structure of a complex of the Wnt11 gene product and its target molecule (e.g., a receptor) can be examined for evidence of interactions, for example the fit of a peptide side chain into a cleft of the target molecule, potential sites for hydrogen bonding, etc. The spatial arrangements of the pharmacophoric groups can be determined by NMR spectroscopy or X-ray diffraction studies. An initial three-dimensional model can be refined by energy minimization and molecular dynamics simulation. A template for modeling can be selected by reference to a template database and will typically allow the mounting of 2-8 pharmacophores. A small molecule mimetic is identified wherein addition of the pharmacophoric groups to the template maintains their spatial arrangement as in the Wnt11 gene product.

A small molecule mimetic can also be identified by assigning a hashed bitmap structural fingerprint to the Wnt11 gene product based on its chemical structure, and determining the similarity of that fingerprint to that of each compound in a broad chemical database. The fingerprints can be determined using fingerprinting software commercially distributed for that purpose by Daylight Chemical Information Systems, Inc. (Mission Viejo, Calif., United States of America) according to the vendor's instructions. Representative databases include but are not limited to SPREI'95 (InfoChem GmbH of Munich, Germany), Index Chemicus (ISI of Philadelphia, Pa., United States of America), World Drug Index (Derwent of London, United Kingdom), TSCA93 (United States Environmental Protection Agency), MedChem (Biobyte of Claremont, Calif., United States of America), Maybridge Organic Chemical Catalog (Maybridge of Cornwall, England), Available Chemicals Directory (MDL Information Systems of San Leandro, Calif., United States of America), NCI96 (United States National Cancer Institute), Asinex Catalog of Organic Compounds (Asinex Ltd. of Moscow, Russia), and NP (InterBioScreen Ltd. of Moscow, Russia). A small molecule mimetic of a reference Wnt11 gene product is selected as comprising a fingerprint with a similarity (Tanamoto coefficient) of in some embodiments at least 0.85 relative to the fingerprint of the reference Wnt11 gene product. Such small molecule mimetics can be tested for bonding to an irradiated tumor using the methods disclosed herein.

Additional techniques for the design and preparation of small molecule mimetics can be found in U.S. Pat. Nos. 5,811,392; 5,811,512; 5,578,629; 5,817,879; 5,817,757; and 5,811,515.

Any small molecule or peptide mimetic of the presently disclosed subject matter can be used in the form of a pharmaceutically acceptable salt. Suitable acids which are capable for use with the small molecule or peptide mimetics of the presently disclosed subject matter include, but are not limited to inorganic acids such as trifluoroacetic acid (TFA), hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid, or the like.

Suitable bases capable of forming salts with the small molecule or peptide mimetics of the presently disclosed subject matter include, but are not limited to inorganic bases such as sodium hydroxide, ammonium hydroxide, potassium hydroxide and the like; and organic bases such as mono-di- and tri-alkyl and aryl amines (e.g. triethylamine, diisopropyl amine, methyl amine, dimethyl amine and the like), and optionally substituted ethanolamines (e.g. ethanolamine, diethanolamine and the like).

As used herein, the phrase “cardiomyogenic differentiation-inducing amount” refers to an amount of a Wnt11 gene product that when present within an in vitro differentiation medium, causes a BMMNC to differentiate into any cell type of the cardiomyogenic lineage.

As disclosed herein, contacting cells that are competent to differentiate along the cardiomyogenic lineage with a Wnt11 gene product induces such differentiation. The contacting can be performed using any method of preparation that comprises a Wnt11 gene product. In some embodiments, a purified Wnt11 gene product (e.g., a recombinantly produced Wnt11 polypeptide) is added to a medium in which the BMMNCs are growing. The addition of the Wnt11 polypeptide can be by direct addition or by co-culture of the BMMNCs with a cell or cell line that produces the Wnt11 gene product and introduces it into the culture medium. In some embodiments, the Wnt11 expressing cell is a cell that has been transformed to comprise an expression vector encoding a Wnt11 gene product (e.g., an expression vector encoding a human Wnt11 gene product or a functional fragment thereof).

In some embodiments, the contacting step is accomplished without contacting the plurality of BMMNCs with any cells or cell types other than

BMMNCs. For example, in the co-culture referenced hereinabove, it is possible to have a physical barrier that separates the BMMNCs in culture from any other cells, including but not limited to the cells that express the Wnt11 gene product. In some embodiments, the cells that express the Wnt11 gene product secrete the Wnt11 gene product into the medium in which they are growing to thereby condition the medium. The conditioned medium is then employed as the growth medium for the BMMNCs, allowing the Wnt11 gene product present therein to contact the BMMNCs.

The contacting step is allowed to proceed for a time and under conditions sufficient to induce cardiomyogenic differentiation in at least a subset of the

BMMNCs. An exemplary set of conditions that are sufficient to induce cardiomyogenic differentiation in at least a subset of the BMMNCs comprises that amount of time and those conditions that induce expression of at least one gene selected from the group including, but not limited to cardiac troponin T (cTnT), cardiac myosin heavy chain (cMyHC)], and connexin 43. Methods for assaying expression of these indicators of cardiomyogenic differentiation are known in the art and include assaying induction of expression at the RNA level, the protein level, or both. In some embodiments, the time and conditions include culturing the BMMNCs in Wnt11-conditioned medium for at least 3, 5, 7, 10, 14, 17, or 21 days, or for any period of time there between. An exemplary time period is at least 21 days.

### IV. Methods and Compositions for Treatment Using Wnt11-Induced BMMNCs

The presently disclosed subject matter also provides treating an injury to cardiac tissue in a subject, the method comprising administering to the subject a composition comprising a plurality of Wnt11-induced bone marrow mononuclear cells (BMMNCs) in a pharmaceutically acceptable carrier, in an amount and via a route sufficient to allow at least a fraction of the plurality of Wnt11-induced BMMNCs to engraft the cardiac tissue, whereby the injury is treated.

As used herein, the phrase “treating an injury to a cardiac tissue in a subject” refers to both intervention designed to ameliorate the symptoms of causes of the injury in a subject (e.g., after initiation of the disease process) as well as to interventions that are designed to prevent the disease from occurring in the subject. Stated another way, the term “treating”, and grammatical variants thereof, is intended to be interpreted broadly to encompass meanings that refer to reducing the severity of and/or to curing a disease, as well as meanings that refer to prophylaxis. In this latter respect, “treating” refers to “preventing” or otherwise enhancing the ability of the subject to resist the disease process.

IV.A. Formulations

The compositions of the presently disclosed subject matter comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

IV.B. Administration

Suitable methods for administration the cells of the presently disclosed subject matter include, but are not limited to intravenous administration and delivery directly to the target tissue or organ. In some embodiments, the method of administration encompasses features for regionalized delivery or accumulation of the cells at the site in need of treatment. In some embodiments, the cells are delivered directly into the tissue or organ to be treated.

As is known in the art, differences in major histocompatability antigens between a transplanted cell or tissue and the host into which it is transplanted can lead to rejection of the transplant by the host (host versus graft rejection) and/or rejection of the host by the transplant (graft versus host rejection). If either is likely to be experienced by a subject subsequent to transfer of the Wnt11-induced cells into the subject, immunosuppressive measures that are standard in the art of transplantation medicine can be employed.

IV.C. Dose

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using the assay methods described herein, one skilled in the art can readily assess the potency and efficacy of a candidate compound of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

### V. Screening Methods

In some embodiments, the presently disclosed subject matter also provides methods for screening candidate compounds for an ability to induce cardiomyogenic differentiation in cells that are competent for differentiating into cardiomyocytes and/or their precursors. In some embodiments, the methods comprise screening candidate compounds for an ability to induce cardiomyogenic differentiation in unfractionated, density gradient-separated bone marrow mononuclear cells (BMMNCs).

In some embodiments, a candidate compound is provided in the form of a library of candidate molecules. As used herein, the term “library” means a collection of molecules. A library can contain a few or a large number of different molecules, varying from about ten molecules to several billion molecules or more. A molecule can comprise a naturally occurring molecule, or a synthetic molecule, which is not found in nature. Optionally, a plurality of different libraries can be employed simultaneously for screening.

Representative libraries include but are not limited to a peptide library (U.S. Pat. Nos. 6,156,511, 6,107,059, 5,922,545, and 5,223,409), an oligomer library (U.S. Patent Nos. 5,650,489 and 5,858,670), an aptamer library (U.S. Pat. Nos. 6,180,348 and 5,756,291), a small molecule library (U.S. Pat. Nos. 6,168,912 and 5,738,996), a library of antibodies or antibody fragments (U.S. Pat. Nos. 6,174,708, 6,057,098, 5,922,254, 5,840,479, 5,780,225, 5,702,892, and 5,667,988), a library of nucleic acid-protein fusions (U.S. Pat. No. 6,214,553), and a library of any other affinity agent that can potentially bind mimic the cardiogenic differentiation-inducing activity of a Wnt11 gene product. (e.g., U.S. Pat. Nos. 5,948,635, 5,747,334, and 5,498,538).

The molecules of a library can be produced in vitro, or they can be synthesized in vivo, for example by expression of a molecule in vivo. Also, the molecules of a library can be displayed on any relevant support, for example, on bacterial pili or on phage. Techniques for generating phage-displayed libraries are known in the art.

A library can comprise a random collection of molecules. Alternatively, a library can comprise a collection of molecules having a bias for a particular sequence, structure, or conformation. See e.g., U.S. Pat. Nos. 5,264,563 and 5,824,483. Methods for preparing libraries containing diverse populations of various types of molecules are known in the art, for example as described in U.S. Patents cited herein above. Numerous libraries are also commercially available.

### VI. Methods for Monitoring Gene Expression During Cardiomyogenic Differentiation

The presently disclosed subject matter also provides methods for monitoring gene expression during cardiomyogenic differentiation. In some embodiments, the methods comprise contacting a plurality of BMMNCs with a cardiomyogenic differentiation-inducing amount of a Wnt11 gene product and assaying gene expression of one or more genes in that subset of the BMMNCs that initiate cardiomyogenic differentiation. Methods for assaying gene expression differences can include a variety of nucleic acid detection assays to detect or quantitate the expression level of a gene or multiple genes in a given sample. For example, Northern blotting, nuclease protection, RT-PCR (e.g., quantitative RT-PCR; QRT-PCR), and/or differential display methods can be used for detecting gene expression levels. In some embodiments, methods and assays of the presently disclosed subject matter are employed with array or chip hybridization-based methods for detecting differential expression of a plurality of genes.

Thus, in some embodiments the assaying comprises comparing gene expression of a plurality of genes in the subset of BMMNCs using an oligonucleotide array. Methods for assaying changes in gene expression using oligonucleotide arrays are known in the art.

In some embodiments, gene expression changes are assayed by removing a subset of cells that have been contacted with a cardiomyogenic differentiation-inducing amount of a Wnt11 gene product at various times after the induction of differentiation and assaying the expression of one, several, or many genes in the various samples.

In some embodiments, preliminary experiments assaying expression of one or more genes (e.g., assaying global or near global gene expression in a given cell using a microarray) can identify one or more genes for which expression can be shown to change during the process of cardiomyogenic differentiation. When genes of interest are identified and deemed to be of particular interest and/or importance, it is possible to employ additional strategies that concentrate on assaying expression changes in just these genes. For example, quantitative RT-PCR can be employed using primers that are designed to specifically bind to RNA molecules transcribed from these genes.

### VII. Cardiomyoqenic Induction Systems

The presently disclosed subject matter also provides systems for inducing cardiomyogenic differentiation in a cell of interest. The disclosed systems include a source of a Wnt11 polypeptide or a functional fragment thereof a growth area for the cell of interest, and a barrier that physically separates the source of the Wnt11 polypeptide from the cultured cell that is permeable to the Wnt11 polypeptide, thereby allowing the Wnt11 polypeptide provided by the source to contact the cultured cell. In some embodiments, the barrier comprises a membrane that is permeable to the Wnt 11 polypeptide or the functional fragment thereof.

Any source of a Wnt11 polypeptide or a functional fragment thereof can be employed. In some embodiments, the source of the Wnt11 polypeptide comprises a second cell that expresses a secretable Wnt11 polypeptide, and the barrier prevents physical contact between the second cell that expresses the secretable Wnt11 polypeptide and the cultured cell in which cardiomyogenic differentiation is to be induced. In some embodiments, the second cell is a recombinant cell that comprises an expression vector encoding the secretable Wnt11 polypeptide.

Additionally, any Wnt11 polypeptide or functional fragment thereof can be employed to induce cardiomyogenic differentiation. In some embodiments, the Wnt11 polypeptide comprises an amino acid sequence including, but not limited to:
(a) amino acids 1-354 of GENBANK® Accession No. P51891;
(b) amino acids 1-354 of GENBANK® Accession No. NP_{—}004617;
(c) a functional fragment of (a) or (b); or
(d) an amino acid sequence at least 95% identical to either (a) or (b),
wherein the Wnt11 polypeptide induces cardiomyogenic differentiation in the cultured cell.
   Mixtures comprises various combinations of (a)-(d) can also be employed.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Materials and Methods Used in the Examples

The experiments disclosed herein were performed in accordance with the guidelines of the Animal Care and Use Committee of the University of Louisville School of Medicine, Louisville, Ky., United States of America, and with the Guide for the Care and Use of Laboratory Animals (United States Department of Health and Human Services, Publication No. [NIH] 86-23).

Animals. Adult C57BL/6 mice (age 6-10 wk, body wt 20-25 g) were used for this study. Donor mice for each experimental group were obtained from the same litter.

Generation of stably transfected Wnt11/293 cell sublines. All tissue culture media and related reagents use for transfection were purchased from Invitrogen Corp. (Carlsbad, Calif., United States of America). HEK-293 cells (a permanent cell-line) were expanded in CD-293 medium supplemented with GLUTAMAX™ (4 μM/ml) and 10% fetal bovine serum (FBS) in a humidified incubator at 37° C. in an atmosphere of 5% CO₂ until 95% confluence was reached.

The Wnt11 expression vector (pWnt11/cDNA3) employed herein was a gracious gift from Dr. Leonard M. Eisenberg and the details of its construction have been previously described (Eisenberg et al., 1997). Briefly, the construct consists of full-length quail Wnt11 complementary DNA (cDNA) inserted immediately downstream of the cytomegalovirus promoter in the eukaryotic expression vector pcDNA3 (Invitrogen Corp., Carlsbad, Calif., United States of America) containing both neomycin and ampicillin resistance genes. pWnt11/cDNA3 or pcDNA3 (empty vector) vectors were used to transfect HEK-293 cells. Stable transfection of HEK-293 cells containing pWnt11/cDNA3 or empty vector (pcDNA3) was accomplished using 1-3 μg of vector DNA introduced into 293 cells using the LIPOFECTAMINE™ transfection reagent according to the manufacturer's protocol. After culturing 293 cells in the presence of 500 μg/mL of Geneticin (G418) for 5 weeks, clones of 293 cells with stable incorporation of the Wnt11 transgene or the pcDNA3 plasmid were selected based upon their resistance to neomycin and designated as Wnt11/293-1, Wnt11/293-2, and empty-vector (pcDNA/293) cells lines, respectively. To maintain expression of the Wnt11 transgene and to ensure stable transfection of vector control, all three of these clones were continuously passaged (every fourth day) in the presence of G418 for an additional 5 weeks.

To verify Wnt11 protein expression by Wnt11/293 cell lines, cultures from each cell line were allowed to reach 90% confluence and analyzed by Western blotting as described hereinbelow. To verify the presence of Wnt11 protein in the media of Wnt11/293 cultures, Wnt11-conditioned media (CM; Wnt11-CM) was harvested from cultures of Wnt11/293 cells grown in 0.5% albumin for 48 hours and then switched to serum-free media once 90% confluent. The presence of Wnt11 protein within CM was verified by Western blot analysis as described hereinbelow and as described previously (Brandon et al., 2000). Briefly, the medium containing Wnt11 was treated with 50 μg/mL of unfractionated heparin (which solubilizes Wnt11 protein bound to cell surfaces and matrix) for 24 hours in a humidified incubator in an atmosphere of 5% CO₂ at 37° C. Media was then collected and assayed for Wnt11 protein expression by immunoblotting following concentration of Wnt11 protein using NANOSEP® centrifugal concentrating devices (Pall Life Sciences, East Hills, N.Y., United States of America), in accordance with the manufacturer's protocol.

Isolation and culture of unfractionated BM-derived mononuclear cells (BMMNCs). For each experiment, two mice were euthanized by CO₂ inhalation followed by cervical dislocation and their femurs and tibias were removed and placed in Dulbecco's Modified Eagle Medium/F12 (DMEM/F12; Invitrogen Corp.). The bones were stripped of soft tissue with fine forceps exposing the long bone shafts. The BM was flushed into and suspended in fresh DMEM/F12 using a 23-gauge needle and 5-ml syringe. The BM was further dispersed by repeated passage through the 23-gauge needle for 10 minutes.

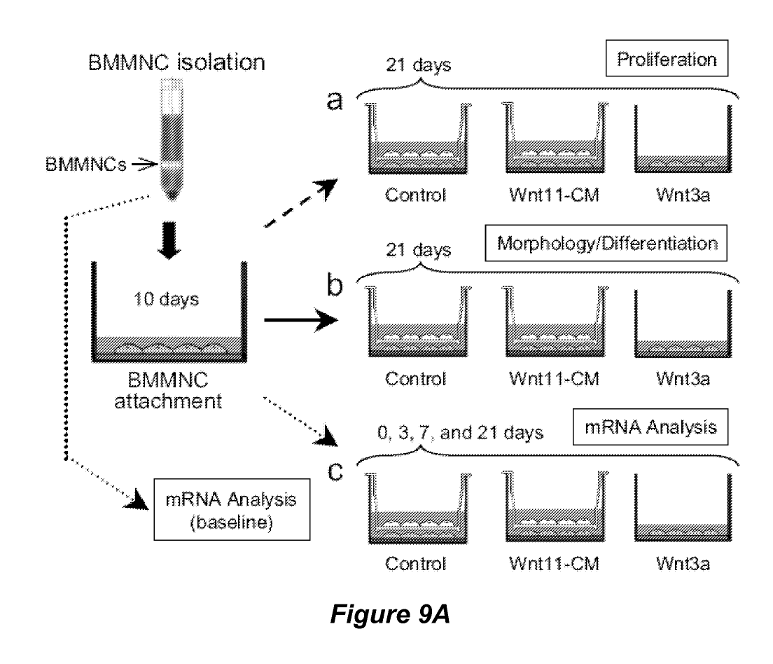

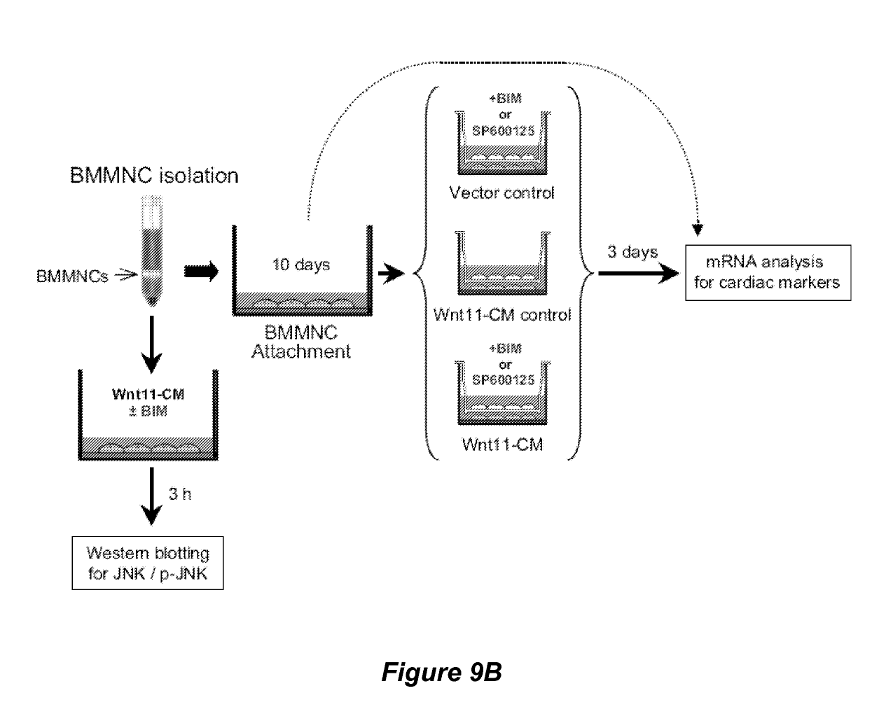

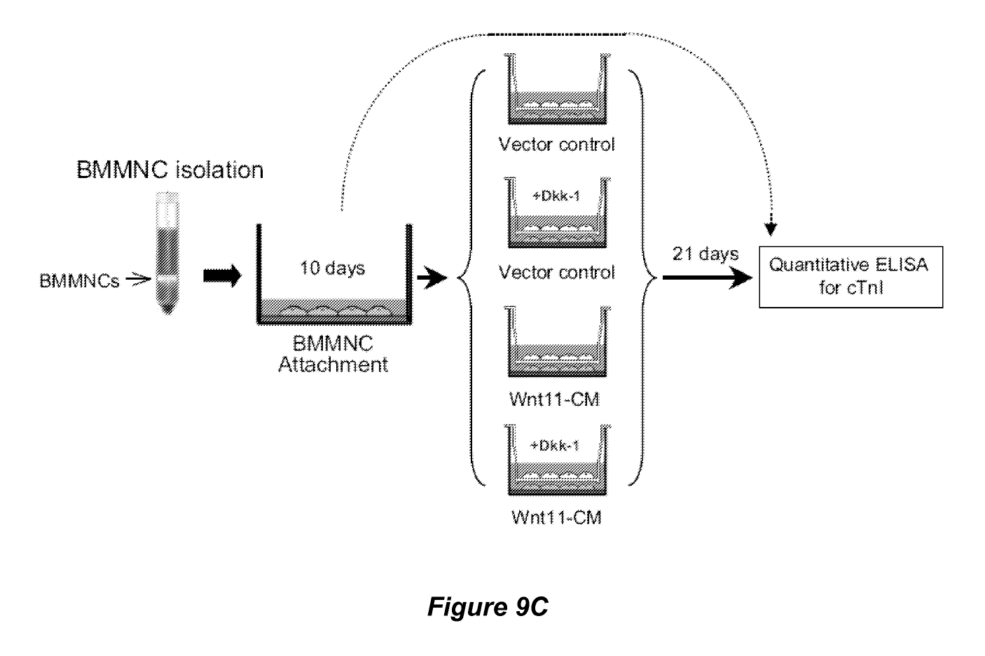

After filtering cells through a 70 μm nylon mesh filter, the MNC fraction (BMMNCs) were separated using FICOLL-PAQUE™ (Sigma Aldrich, St. Louis, Mo., United States of America) density-gradient centrifugation as described in (Zuba-Surma et al., 2006). See also FIG. 9A. The viability of unfractionated BMMNCs was assessed by Trypan blue (Sigma Aldrich) staining. Approximately 15×10⁶ unfractionated BMMNCs were obtained from each mouse. Adult unfractionated BMMNCs were seeded (n=5×10⁵ cells/well) in 6-well tissue culture plates (lower chamber of a 6-well tissue culture TRANSWELL® system; Corning Inc. Life Sciences, Lowell, Mass., United States of America) on fibronectin (FN)-coated glass coverslips (Fischer Scientific). To ensure maximal attachment of BMMNCs, cultures were maintained in complete medium containing DMEM/F12, supplemented with 100 IU/mL penicillin G/streptomycin (GIBCO®, a division of Invitrogen Corp.) and 10% fetal bovine serum (FBS; GIBCO®) for 10 days in a humidified incubator in an atmosphere of 5% CO₂ at 37° C. (see FIG. 9A). A single medium change was performed at day 6 and unattached cells were carefully drawn off, pelleted, and added back to attached fraction with fresh complete medium. Attachment of all BMMNC subfractions was complete by day 10, whereupon very few cells remained unattached. Medium was changed once again and replaced with complete medium.

Induction of differentiation. To assess the influence of ectopic expression of Wnt11 on BMMNC differentiation, Wnt11 conditioned medium (Wnt11-CM) was generated. Specifically, unfractionated BMMNCs were co-cultured with Wnt11-expressing HEK-293 cells (Wnt11/293-2; designated as Wnt11-CM), under the same conditions used to culture BMMNCs. In order to achieve strict separation of cell types during co-culture experiments, BMMNC and Wnt11/293 cells were separated into upper and lower chambers of a TRANSWELL® co-culture system. Wnt11/293-2 or pcDNA3/293 (empty vector) cells (the controls in this experiment) were cultured in the upper chamber on the polyester (PET) membrane of a 24 mm TRANSWELL® insert, and unfractionated BMMNCs were cultured in the lower chamber as described above.

Moreover, to eliminate the possibility of cell fusion or cell-cell contact as a requirement for Wnt11-mediated effects, a membrane pore size of 0.4 μm was chosen, thus preventing egression of Wnt11/293-2 cells to the lower compartment that contained BMMNCs. Of note, TRANSWELL® inserts were maintained separately without cells until day 10 of BMMNC attachment. On day 10: (1) Wnt11/293-2 or pcDNA3/293 (control) cells were added to the inserts; (2) inserts containing Wnt11/293-2 cells were placed atop cultured BMMNCs present in the lower chambers (Wnt11-CM, as defined above) and inserts containing pcDNA3/293 cells were placed atop cultured BMMNCs present in the lower chambers of other plates (designated as control medium; see FIG. 9A). Cells were cultured for 21 days with medium change every fourth day.

Additionally, to assess the influence of Wnt3a on cellular differentiation, unfractionated BMMNCs were cultured separately in the presence of 150 ng/ml of recombinant Wnt3a protein under the same conditions used to culture unfractionated BMMNCs, using pcDNA3/293 cells containing the empty vector as controls (see FIG. 9A).

Cytomorphological evaluation of cell-types. Transmission confocal micrographs were evaluated for cellular morphology in a blinded manner by a cytopathologist. Differentiation status was defined grossly based on early blast-like (myeloblastic and promyelocytic) stages of hematopoiesis, later granular stages (myelocytes and metamyelocytes) and late granulocytic and phagocytic stages. Endothelial, epithelial, or fibroblastic morphology was also delineated.

Assessment of cellular proliferation. To study proliferation within each experimental group, unfractionated BMMNCs (n=1×10⁴) were cultured as above in separate experiments (see FIG. 9A). Cells were monitored by phase contrast microscopy during culture in a humidified 5% CO₂ incubator at 37° C. for up to 21 days. The medium was replenished every 6 days and expanded cells were harvested by trypsinization and gentle resuspension on days 8, 16, and 21. Cell number and viability was determined by Trypan blue staining, and the fold-increase in cell number over time was analyzed.

Immunocytochemistry. Cells were fixed on glass cover-slides with 4% paraformaldehyde for 20 minutes at 4° C. Cells were washed four times and permeabilized with BD PERM/WASH™ (containing saponin; BD Biosciences, San Jose, Calif., United States of America). For immunostaining, separate preparations of BD PERM/WASH™ containing primary monoclonal antibodies against either cardiac troponin T (cTnT; Santa Cruz Biotechnology, Santa Cruz, Calif., United States of America) or cardiac myosin heavy chain (cMyHC; Novus Biologicals, Inc., Littleton, Colo., United States of America) or connexin-43 or Oct-4 were prepared; all primary antibodies, save cMyHC, were purchased from Santa Cruz Biotechnology. Each preparation also contained primary monoclonal antibodies against nestin, glial fibrillary acidic protein (GFAP), CD11B, Gr-1 (myeloid lineage markers), the CD45 epitope, myogenin

(Santa Cruz Biotechnology) and others (see Zuba-Surma et al., 2006).

These preparations were then applied separately (i.e., mixtures either with cTnT or with cMyHC) or, for co-immunostaining experiments, in combinations to cells for 16 hours at 4° C. and 37° C. for the final hour. Subsequently, cells were washed another five times in BD PERM/WASH™ and then rhodamine (TRITC)-conjugated or fluorescein isothiocyanate (FITC)-conjugated secondary antibodies (Jackson ImmunoResearch Laboratories, Inc., West Grove, Pa., United States of America) were applied to cells for two hours at 4° C. to visualize expression of cardiac specific proteins. DAPI (Invitrogen—MOLECULAR PROBES®, Eugene, Oreg.) was added in the final 10 minutes of secondary antibody incubation for nuclei visualization. Photomicrographs of cells were obtained using a LSM 510 (Carl Zeiss, Inc., Thornwood, N.Y., United States of America) inverted laser scanning confocal microscope.

Assessment of cardiac differentiation. For quantitative assessment of cardiomyocytic differentiation, an average of 2400 cells/plate were counted. Multiple fields from all 4 quarters of the plate were systematically evaluated. The number of cells expressing cardiac-specific intracellular contractile proteins in each field was counted; the total number of cells was calculated based on nuclear staining with DAPI. In addition, cells were concomitantly stained with markers specific for various non-cardiac lineages (skeletal muscle [myogenin]; neural [nestin, GFAP]; myeloid [CD11b, Gr-1]; and others; Zuba-Surma et al., 2006). Cells were counted as positive when they were positive for cardiac-specific contractile proteins and negative for all of these markers. Thus, a rigorous method was employed to assess cardiac differentiation in vitro. The percentage of cells differentiating into cardiomyocytes was calculated as the number of cells positive for proteins divided by the total number of nuclei in the field. These experiments were performed in triplicate. As a confirmatory measure, the expression of cardiac-specific troponin I (cTnI) was determined by ELISA as described hereinbelow following culture in Wnt11-CM with and without the Wnt inhibitor Dickkopf-1 (Dkk-1; see FIG. 9C).

Assessment of mRNA expression by quantitative real-time RT-PCR (qRT-PCR). All qRT-PCR reagents and primers were purchased from Qiagen Inc. (Valencia, Calif., United States of America). Prior to mRNA extraction, cell number was determined and calculated by Trypan blue exclusion. cDNA was generated from freshly isolated (designated as baseline) and cultured unfractionated BMMNCs at days 0, 3, 7, and 21 (see FIG. 9A) using QUANTISCRIPT REVERSE TRANSCRIPTASE™ (Qiagen Inc., Valencia, Calif., United States of America) contained in RT master mix of Cell to cDNA kits according to manufacturer's protocol. Each experiment contained two negative controls: (1) H₂O alone; and (2) a total RNA sample prepared in the absence of the reverse transcriptase enzyme (-RT). At 0, 3, 7, and 21 days, cell monolayers were collected for cDNA preparation. For qRT-PCR, validated forward and reverse primer sets for the following genes were obtained from cDNA sequences contained in the public GENBANK® sequence database of the United States National Center for Biotechnology Information: α-MyHC, β-MyHc, cTnT, Nk×2.5, GATA-4, ANP, β-catenin, Oct-4, Nanog and 18S rRNA.

To avoid amplification of contaminating DNA, all validated qRT-PCR primers were designed to cross intron-intron sequences within the cDNA to be amplified. Quantitative qRT-PCR analysis was performed in an ABI Prism 7900H sequence detection system (Applied Biosystems, Foster City, Calif.) using QUANTITECT™ SYBR® Green Real Time PCR kits. Each 50 μl qRT-PCR reaction contained 2 μl (approximately 50 ng) of cDNA, 5 μl of 1× primer mix (containing approximately 500 nM sense and anti-sense primers) and 25 μl of 2× QUANTITECT™ SYBR® Green master mix. Triplicates of each sample were amplified for 40 cycles.

The PCR product level is calculated from the threshold cycle (Cₜ). That is, the amplification cycle number at which exponential PCR-generated gene-specific fluorescence is first detected (above a fixed threshold level). The ΔCₜ values for each gene were normalized to an internal control ΔCₜ value (obtained from 18s ribosomal mRNA expression) in each sample and expressed as a -fold difference, calculated according to relative quantification method in relation to appropriate calibrators (see Pfaffl, 2001); either baseline BMMNCs mRNA levels or mRNA levels of BMMNCs at day 3 of culture in Wnt11-CM.

Assessment of Wnt signaling. To investigate non-canonical signaling pathways, freshly isolated unfractionated BMMNCs were cultured in Wnt11-CM or in empty vector (control) medium in the presence or absence of 1 μM of the PKC inhibitor bisindolylmaleimide I (BIM, Sigma) for 3 hours followed by harvest and assessment of JNK phosphorylation by Western immunoblotting (see below and FIG. 9B). The expression of cardiac-specific gene expression in unfractionated BMMNCs was assessed by qRT-PCR following culture for 3 days in Wnt11-CM and empty vector (control) medium with and without PKC (BIM; 1 μM) and JNK (SP600125; 10 μM; Sigma) inhibitors (see FIG. 9B). Expression of Wnt3a and β-catenin were measured by qRT-PCR to investigate the effect of control, Wnt3a medium, and Wnt11-CM on canonical signaling.

Western immunoblotting and quantitative ELISA. Western analysis was performed as described in Guo et al., 1998; Brandon et al., 2000; Li et al., 2003; and Li et al., 2006. Briefly, 100 μg of protein was isolated from: (1) the cell lysates of Wnt11/293 cell cultures (alone, without BMMNCs in co-culture) and of Wnt11-treated BMMNC cultures after 3 hours of treatment with BIM (see FIG. 9B); as well as from (2) the Wnt11-conditioned media of Wnt11/293 cells (Wnt11-CM). Isolated proteins were separated on an 8% SDS-polyacrylamide gel and transferred to a nitrocellulose membrane. Gel transfer efficiency was recorded carefully by making photocopies of membranes dyed with reversible Ponceau staining; gel retention was determined by Coomassie blue staining. Membranes were probed with the following antibodies: a specific goat anti-mouse Wnt11 IgG antibody (R&D Systems, Inc., Minneapolis, Minn., United States of America) and rabbit anti-mouse Phospho-SAPK/JNK (Thr183/Tyr185) monoclonal antibody (Cell Signaling Technology, Inc., Danvers, Mass., United States of America). Immunoreactive bands were visualized with horseradish peroxidase-conjugated anti-goat and anti-rabbit IgG (Santa Cruz Biotechnology) using an ECL detection kit (New England Nuclear, Billerica, Mass., United States of America), quantified by densitometry, and normalized GAPDH. In all samples, the content of target protein was expressed as a percentage of the controls (untreated), normalized to GAPDH. The expression of cardiac troponin I (cTnI) in proteins isolated from homogenized cultured cells was quantitated using commercially available ELISA kit (Life Diagnostics, West Chester, Pa., United States of America) according to published methods (see Eckle et al., 2006).

As an additional endpoint to evaluate the influence of a Wnt inhibitor on myocardial protein marker expression, cardiac troponin I (cTnI) levels were measured in BMMNCs cultured for 21 days in the presence or absence of Wnt11-CM, with or without the addition of Dickkopf-1, an inhibitor of canonical Wnt signaling. Briefly, the homogenized proteins were isolated from these cells following 21 days of culture. The cTnI levels were quantitatively determined (as ng of cTnI per mg of protein) with mouse cTnI ELISA kit (Life Diagnostics, West Chester, Pa., United States of America) according to published methods (see Eckle et al., 2006). The cTnI levels were expressed as a percentage of the controls (untreated).

Statistical Analysis. Data are expressed as mean±SEM. Differences were analyzed using the unpaired Student's t-test or ANOVA (one-way or repeated measures with one between-subjects factor [group] and one within-subjects factor [time-point]) as appropriate. Following ANOVA, pairwise comparisons were performed with the Student's t-test using the Bonferroni correction. Statistical significance was defined as P<0.05. When Bonferroni correction was used, the significance level of each individual test was adjusted downwards by dividing 0.05 by the number of possible comparisons. All statistical analyses were performed using the SPSS (version 8.0) statistical software (SPSS Inc., Chicago, Ill., United States of America).

### Example 1

### Generation of Cell Lines with Ectopic Expression of Wnt11

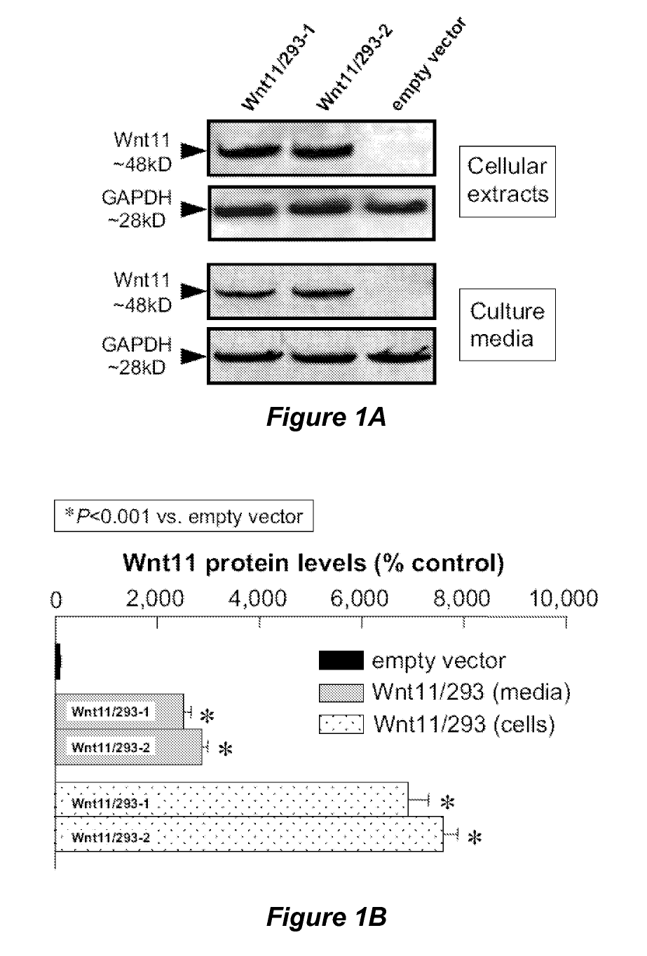

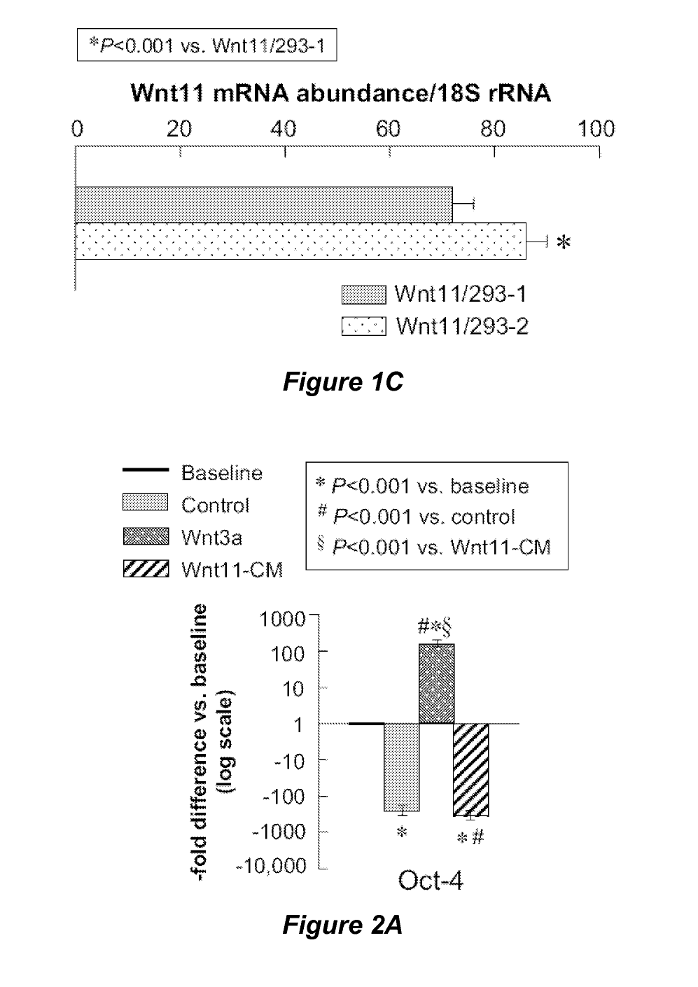

Two Wnt11/293 transfected cell lines (Wnt11/293-1 and Wnt11/293-2) were generated to achieve ectopic overexpression of Wnt11. Both lines demonstrated robust constitutive expression levels of Wnt11 protein in cellular extracts as well as in media, confirming release of Wnt11 from Wnt11/293 cells (FIG. 1A). The protein levels of the active (soluble) form of Wnt11, distinct from the inactive matrix-associated form (Brandon et al., 2000), in the medium were approximately 30% of the cellular levels (FIG. 1B), similar to previous studies that used Wnt11-expression systems (Eisenberg & Eisenberg, 1999). Although Wnt11 expression was higher in Wnt11/293-2 at the mRNA level (FIG. 1C), no significant difference was observed in Wnt11 expression between Wnt11/293-1 and Wnt11/293-2 cells at the protein level (FIG. 1B). Wnt11/293-2 cells were selected for the Wnt11-CM experiments described hereinbelow.

### Example 2

### Morphology of Unfractionated BMMNCs Prior to Culture

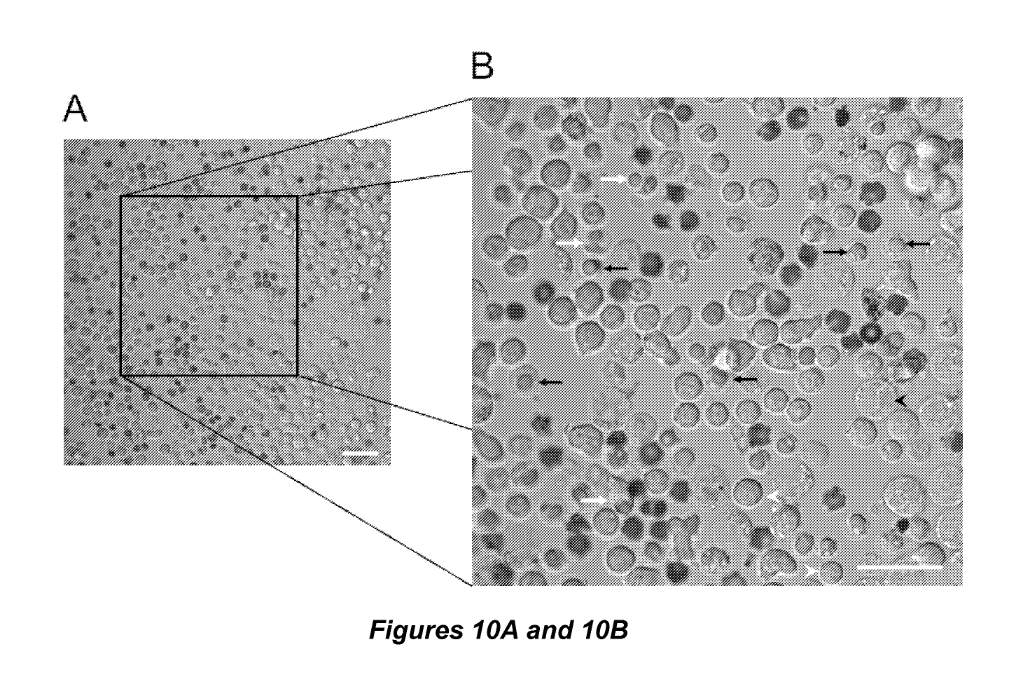

Unfractionated BMMNCs exhibited considerable morphologic variation 24 hours after plating (see FIGS. 10A and 10B). The majority of cells were round and large with eccentric nuclei (promyelocytic precursors) or with a high nuclear:cytoplasmic ratio, characteristic of myeloblastic hematopoietic precursors, whereas a few cells displayed early granulocytic (myelocytic and metamyelocytic) features (Nikolova et al., 2007).

### Example 3

### Effect of Wnt3a and Wnt11 on the Expression of Markers of Pluripotency in Unfractionated BMMNCs in Culture

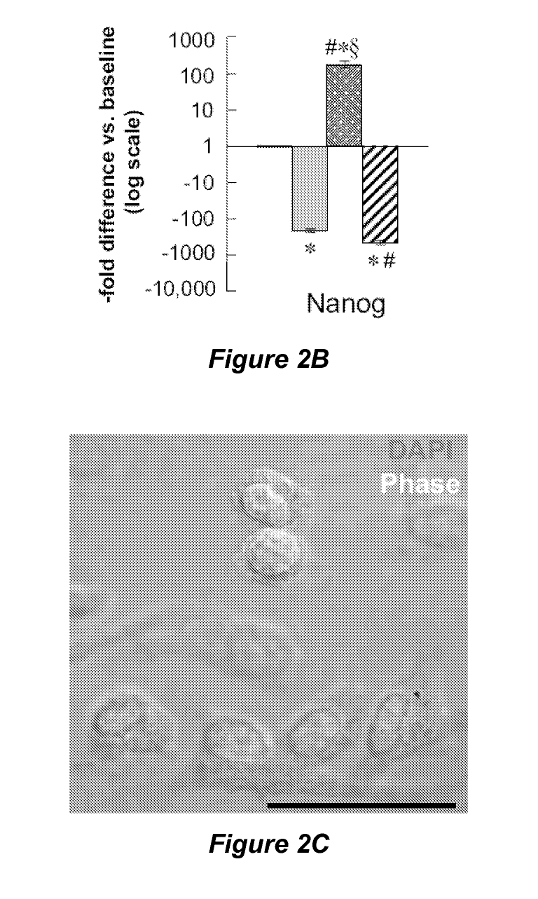

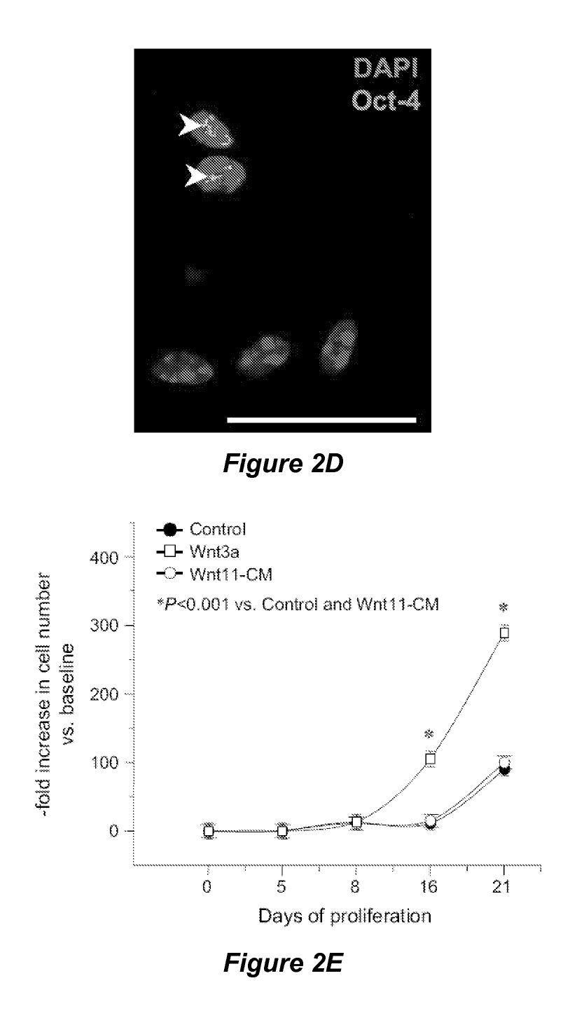

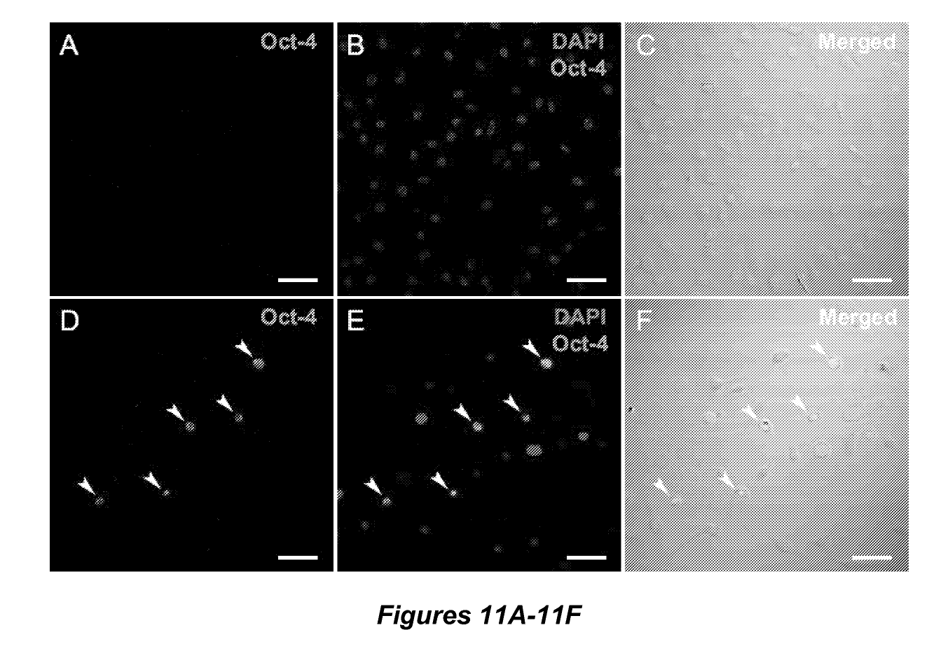

Since Wnt3a and Wnt11 are known to influence both differentiation and renewal of various stem cell populations (Sato et al., 2004; Belema Bedada et al., 2005; Nikolova et al., 2007), the effect of these proteins on the expression patterns of well-known markers of pluripotency that are associated with an undifferentiated state (Oct-4 and Nanog; see e.g., Scholer et al., 1990; Mitsui et al., 2003; Zuba-Surma et al., 2006) was investigated. Culturing BMMNCs for 21 days in control medium exerted a significant negative effect upon the intrinsic pluripotency of these cells, as evidenced by a 264±0.4-fold and 217±0.2-fold decrease (compared with baseline) in the expression of Oct-4 and Nanog, respectively, (P<0.001 for both, FIGS. 2A and 2B). Culturing BMMNCs in Wnt11-CM further enhanced this downregulation of Oct-4 and Nanog expression (352±0.2-fold and 471±0.2-fold decrease, respectively, compared with baseline; P<0.001 for both; FIGS. 2A and 2B). In contrast, Wnt3a markedly enhanced the expression of Oct-4 and Nanog (177±0.4-fold and 158±0.6-fold increase, respectively, compared with baseline; P<0.001 for both; FIGS. 2A and 2B). Moreover, immunocytochemical assessment revealed a marked increase in the number of Oct-4-positive BMMNCs following culture in presence of Wnt3a, compared with untreated control BMMNCs, indicating that Wnt3a increases the number of cells expressing markers of pluripotency (FIGS. 2C, 2D, and 11).

### Example 4

### Effect of Wnt3a and Wnt11 on Proliferation of Unfractionated BMMNCs During Culture

In unfractionated BMMNCs cultured in control medium, only minimal proliferation was noted until the later stages of culture (FIG. 2E). In BMMNCs cultured in Wnt11-CM, the proliferation rate was similar to that in control medium at all time-points. Conversely, starting on day 8, the increase in cell number was significantly greater in BMMNCs cultured in Wnt3a (110±0.1-fold vs. 12±0.1-fold in control and 14±0.1-fold in Wnt11-CM on day 16; P<0.001 for both; 297±0.1-fold vs. 93±0.9-fold in control and 101±0.1-fold in Wnt11-CM on day 21; P<0.001 for both; FIG. 2E), indicating that Wnt3a promotes proliferation of unfractionated BMMNCs.

### Example 5

### Wnt3a Preserves the Hematopoietic Potential of Cultured Unfractionated BMMNCs

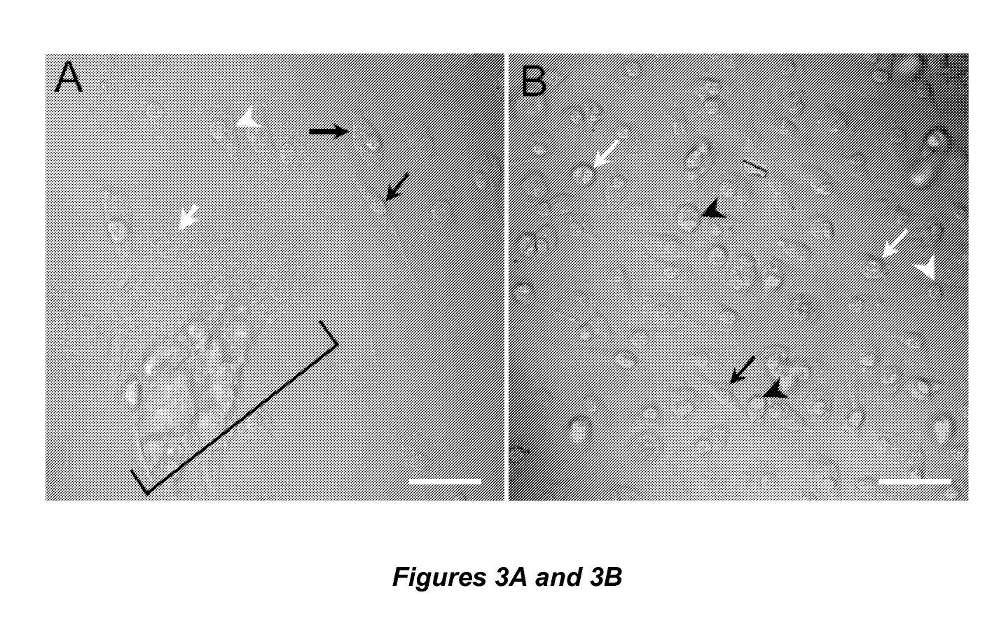

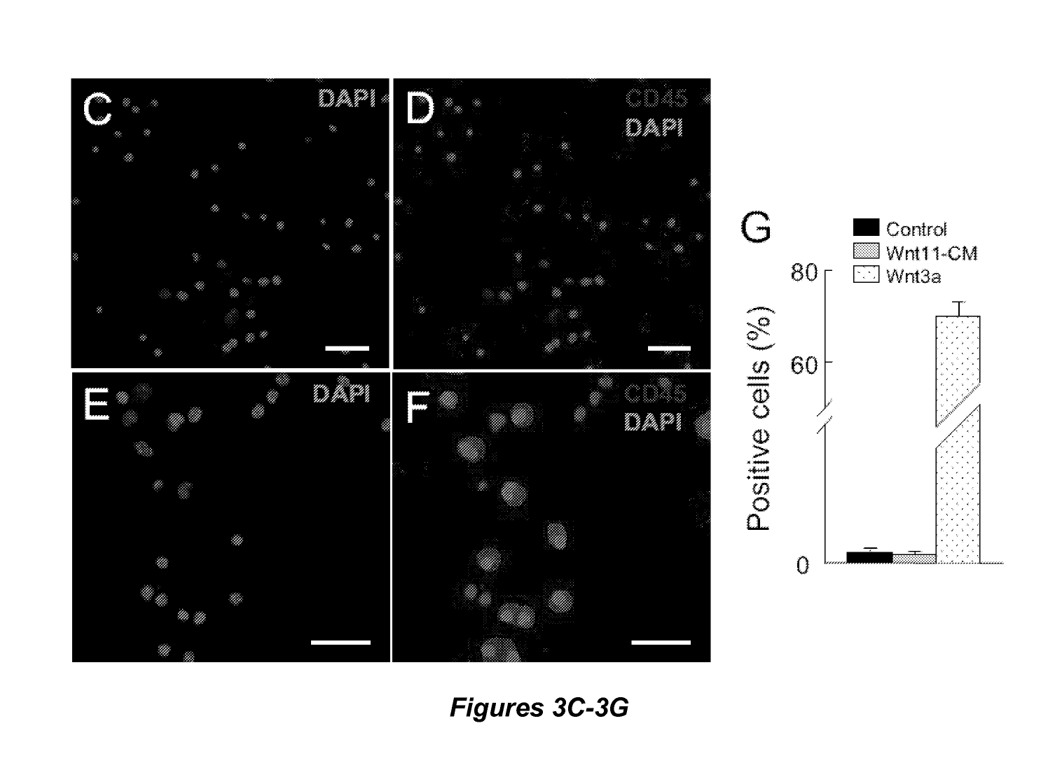

Consistent with the decrease in levels of Oct-4 and Nanog, control BMMNCs displayed considerable morphological heterogeneity during culture (FIG. 3A). In addition, only 0.4±0.1% and 0.3±0.1% of cells in control and Wnt11-CM, respectively, were positive for the pan-hematopoietic marker CD45 after 21 days of culture (FIGS. 3C, 3D, and 3G), indicating attrition of the hematopoietic potential. In contrast, the morphological features of BMMNCs in Wnt3a medium remained largely unaltered during culture (FIG. 3B and FIG. 10), and 69.9±2.9% of these cells expressed CD45 (FIGS. 3E-3G). In conjunction with the data presented above, these results indicate that Wnt3a preserves the hematopoietic potential of unfractionated BMMNCs in culture, which is associated with greater expression of markers of pluripotency. Wnt11 signaling, however, suppresses the markers of pluripotency and promotes cellular differentiation into non-hematopoietic lineages.

### Example 6

### Wnt11 Activates Cardiomyogenesis in Cultured Unfractionated BMMNCs

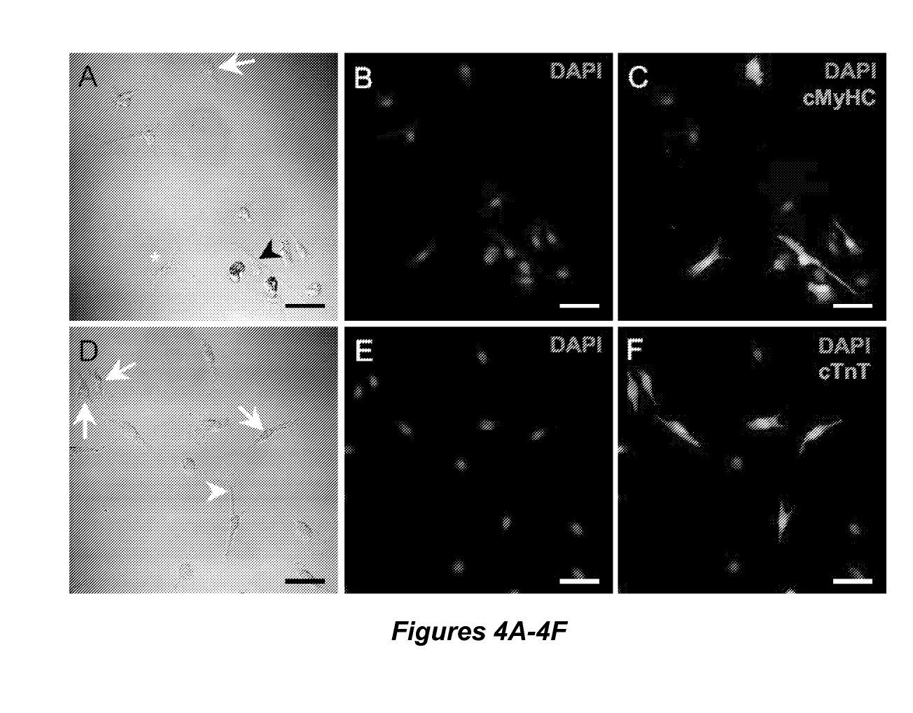

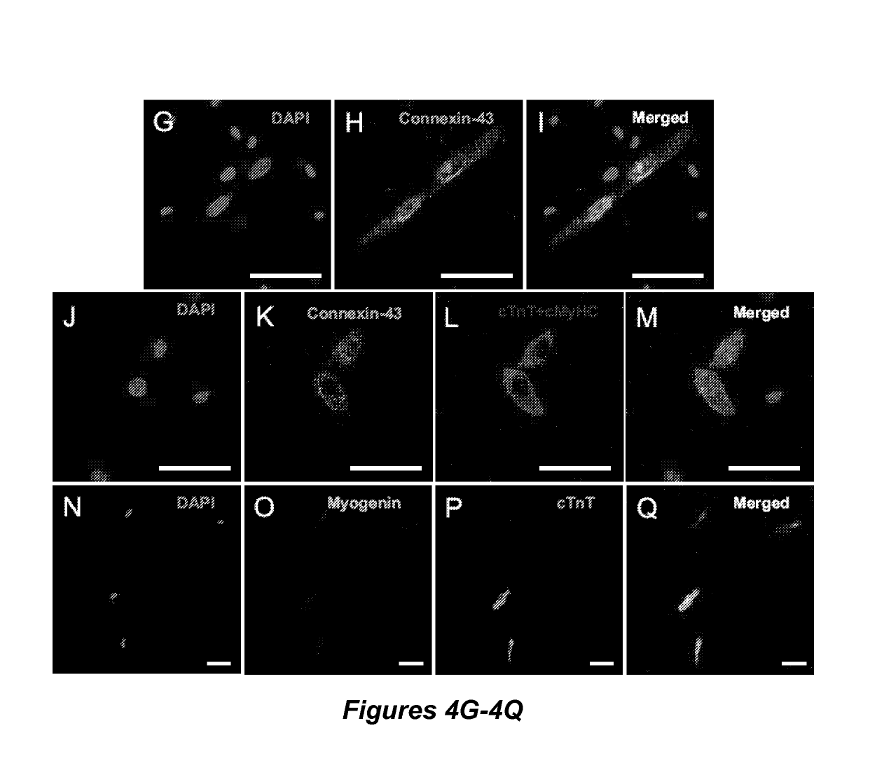

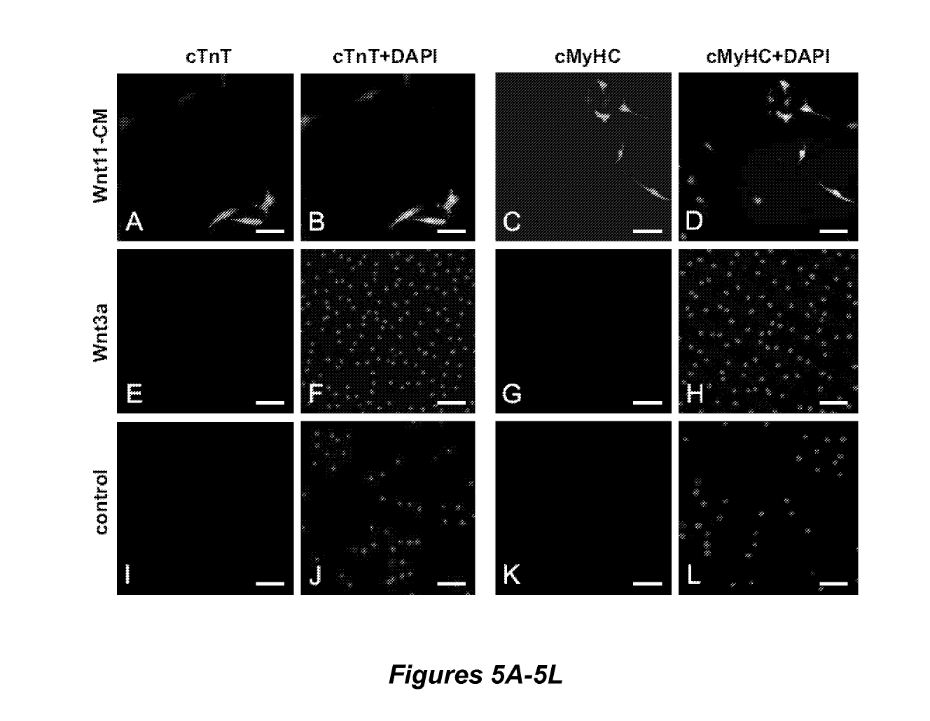

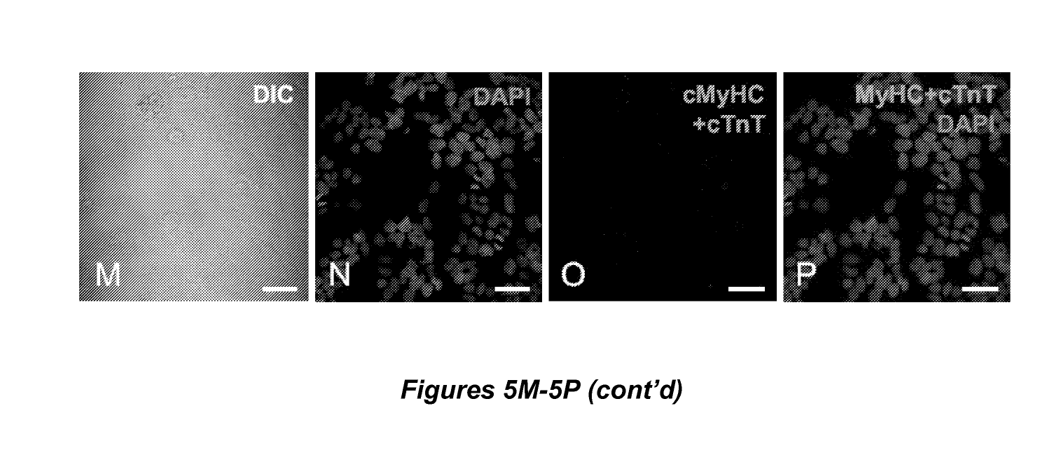

After 21 days of culture in Wnt11-CM, cellular morphology changed from the small and large round cells (FIG. 10) to predominantly elongated, rod-shaped, and elliptical cells (FIGS. 4A and 4D). Immunocytochemical analysis revealed a cardiac phenotype in BMMNCs cultured in Wnt11-CM for 21 days, as evidenced by the positivity for cardiac-specific structural proteins (cMyHC and cTnT; FIGS. 4C and 4F, respectively), connexin-43 (FIGS. 4G-4M), and negativity for markers for non-cardiac lineages, including the skeletal muscle marker myogenin (FIGS. 4N-4Q) and others (Zuba-Surma et al., 2006). Interestingly, although BMMNCs expressed cardiac-specific antigens and acquired a cardiomyocytic morphology, a sarcomeric structure was not apparent in our cultures; moreover, the vast majority of these cells remained mononucleated and, for the most part, did not fuse or form networks (FIG. 4), ostensibly indicating a less mature phenotype. Importantly, after 21 days, only BMMNCs cultured in Wnt11-CM demonstrated cardiomyogenic differentiation (FIGS. 5A-5D), with 27.6±0.6% and 29.6±1.4% of all cells staining positive for cMyHC and cTnT, respectively (FIG. 5Q). Wnt11-expressing Wnt11/293-2 cells showed no evidence of cardiomyogenic differentiation (FIGS. 5M-5P), indicating that the Wnt11-induced cardiac differentiation was specific for BMMNCs. Of note, expression of the myeloid lineage markers CD11b and Gr-1 was observed in very small (and similar) numbers of cultured BMMNCs both in Wnt11-CM and control medium.

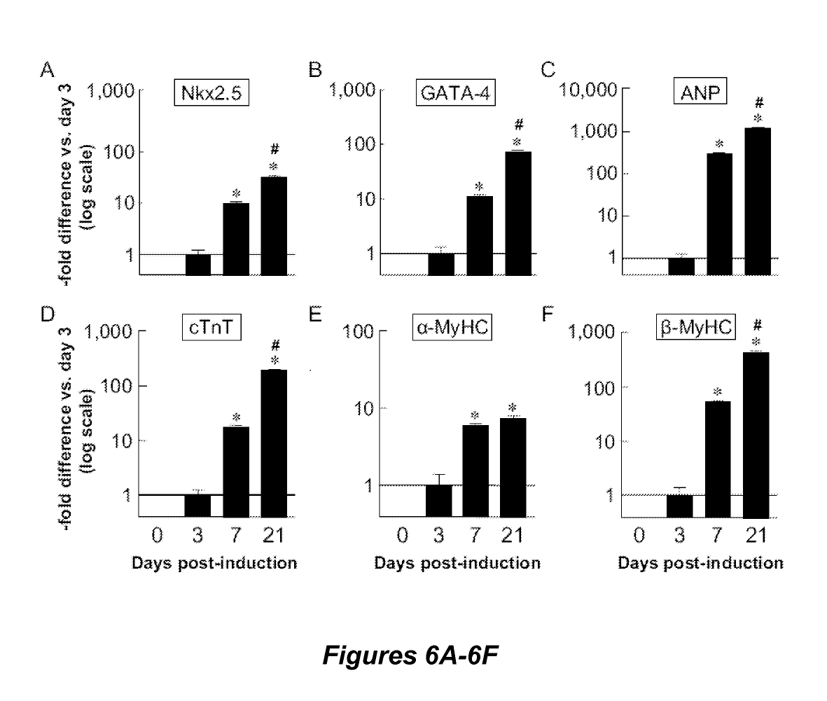

Next, whether Wnt proteins were capable of activating the cardiac gene program in cultured BMMNCs was assessed. qRT-PCR analysis of BMMNCs during 21 days of culture revealed that Wnt11-CM activated the cardiac gene program by day 3 of exposure, as evidenced by expression of early markers of cardiomyogenesis (Nk×2.5, GATA-4, and ANP) and cardiac contractile proteins (cTnT, α-MyHC, and β-MyHC, FIGS. 6A-6F). Notably, no cardiac-specific transcripts were detected in BMMNCs prior to culture in Wnt11-CM (day 0), after 21 days of culture in the presence of the empty vector (pcDNA3-293 cells), or in the presence of Wnt3a. Compared with the levels at day 3, the mRNA levels of cardiac-specific markers at days 7 and 21, respectively, increased as shown in Table 2.

TABLE 2
Fold Increases in Cardiac-specific mRNA Levels

| Marker | Day 7 | Day 21 | P Value |
|---|---|---|---|
| Nkx2.5 | 9.7 ± 0.3 | 31.9 ± 0.4 | <0.001 for both |
| | | | (see FIG. 6A) |
| GATA-4 | 11.1 ± 0.3 | 72.3 ± 0.3 | <0.001 for both |
| | | | (see FIG. 6B) |
| ANP | 293.1 ± 0.3 | 1139.9 ± 0.3 | <0.001 for both |
| | | | (see FIG. 6C) |
| cTnT | 17.8 ± 0.4 | 192.6 ± 0.4 | <0.001 for both |
| | | | (see FIG. 6D) |
| β-MyHC | 51.7 ± 0.3 | 422.6 ± 0.3 | <0.001 for both |
| | | | (see FIG. 6F) |

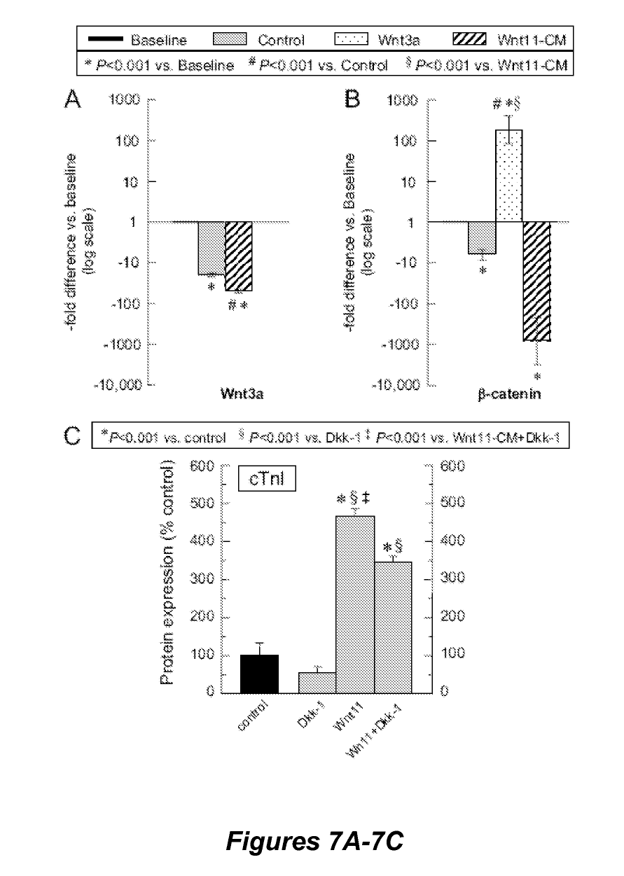

Interestingly, although levels of all transcripts, including β-MyHC, showed a significant progressive increase at each time point, α-MyHC expression increased significantly at day 7 (5.9±0.3-fold; P<0.001; FIG. 6E) but remained essentially unchanged thereafter. This pattern of gene expression is consistent with the temporal sequence of cardiomyogenic gene expression described in embryoid bodies and during fetal development (Makino et al., 1999; Auda-Boucher at al., 2000; Xu et al., 2006). A +367% increase in cTnI expression by ELISA in BMMNCs cultured in Wnt11-CM (compared with untreated controls) was consistent with the immunohistochemical and mRNA findings (FIG. 7C).

### Example 7

### Effect of Wnt Proteins and Wnt Inhibitors on Wnt3a, β-catenin, and cTnI Expression

The β-catenin-mediated canonical pathway, recruited by Wnt3a and other members of the Wnt1 family of molecules, is distinct from the non-canonical signaling pathways used by the Wnt5 family, which includes Wnt11 (Pandur et al., 2002; Nakamura et al., 2003; Belema Bedada et al., 2005; Koyanagi et al., 2005). However, since recent reports have described crosstalk between Wnt11/non-canonical and canonical signaling pathways (Maye et al., 2004; Lev et al., 2005; Ueno et al., 2007), the effect of Wnt proteins on Wnt and β-catenin transcriptional activity in freshly isolated and cultured unfractionated BMMNCs was examined. No Wnt11 expression was detected in freshly isolated or cultured BMMNCs. Consistent with the findings with regard to markers of pluripotency disclosed herein (FIG. 2), culturing in control medium alone was associated with a marked decrease in the expression of both Wnt3a and β-catenin (19.8±0.5-fold and 6.4±0.3-fold decrease, respectively; P<0.001 for both; FIG. 7). Wnt3a and Wnt11-CM exerted opposite effects on the expression of β-catenin in BMMNCs (183.4±0.3-fold increase vs. 859±0.6-fold decrease, respectively; FIG. 7B), similar to the changes observed by others in cultured stem cells (Maye et al., 2004). Interestingly, ectopic overexpression of Wnt11 also modified Wnt3a levels (46±0.1-fold decrease; FIG. 7A).

Next, whether a well-established antagonist of Wnt signaling, Dkk-1 (a soluble inhibitor of canonical signaling that blocks Wnt-signaling at the cell surface), would influence the cardiomyogenic commitment of cultured BMMNCs was examined. BMMNCs were cultured in Wnt11-CM with or without Dkk-1. After 21 days of culture, while Dkk-1 had little effect by itself, it reduced Wnt11-induced expression of cTnI in BMMNCs (FIG. 7C). Together, these data suggested that while Wnt11 attenuated Wnt/β-catenin signaling at the cell surface and at the subcellular level, upstream of β-catenin, and at the level of β-catenin itself, inhibition of canonical signaling diminished the full cardiomyogenic potential of Wnt11/non-canonical signaling.

### Example 8

### Activation of the Cardiac Gene Program is Dependent on Wnt11/PKC Signaling

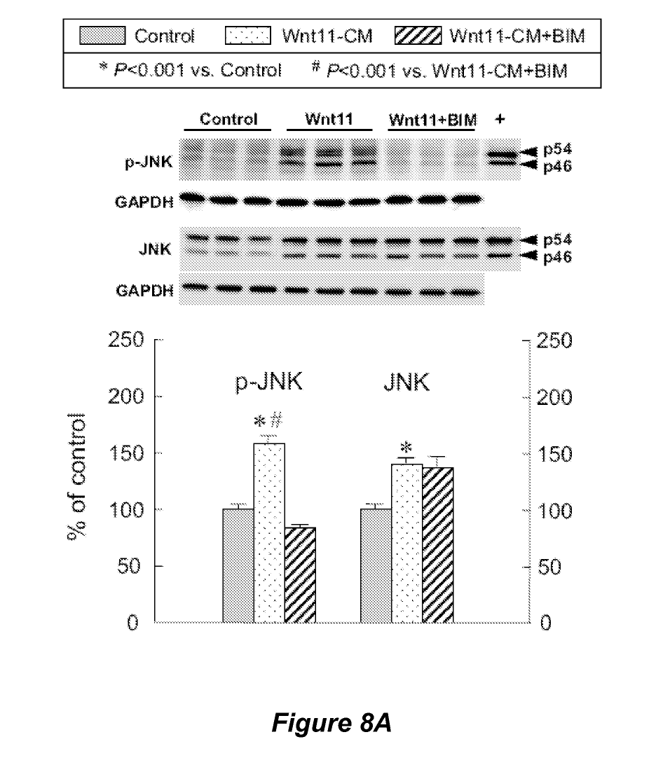

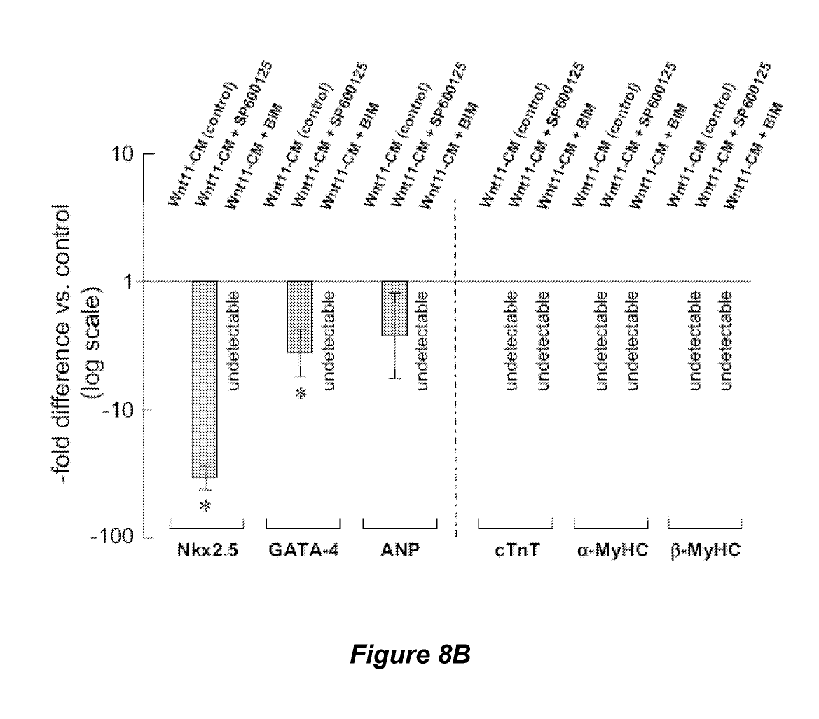

PKC and JNK are generally considered to be the primary mediators of non-canonical Wnt signaling during cardiogenesis (Pandur et al., 2002; Belema Bedada et al., 2005; Koyanagi et al., 2005). Since PKC is upstream of JNK in this signaling pathway (Pandur et al., 2002), the effect(s) of Wnt11-CM on PKC-mediated JNK phosphorylation was tested. Cell viability was estimated to be >98% prior to mRNA expression analysis. No cardiac-specific gene expression was observed in BMMNCs cultured in control (empty vector) medium. Pharmacologic inhibition of PKC with BIM completely blocked Wnt11-induced JNK phosphorylation in BMMNCs (FIG. 8A) and abolished cardiac gene expression (FIG. 8B). Interestingly, although JNK inhibition via SP600125 completely abolished Wnt11-induced expression of cardiac contractile proteins in BMMNCs, the expression of the cardiac transcription factors GATA-4 and Nk×2.5, though significantly depressed, was still detectable and ANP was not significantly affected (FIG. 8B). These data indicate that in unfractionated BMMNCs, Wnt11-activation of cardiomyogenic non-canonical Wnt signaling is completely dependent upon PKC, but only partially dependent upon JNK, suggesting the existence of additional transducing elements in this pathway, likely upstream of JNK and downstream of PKC.

### Example 9

### Intramyocardial Injection of BMMNCs

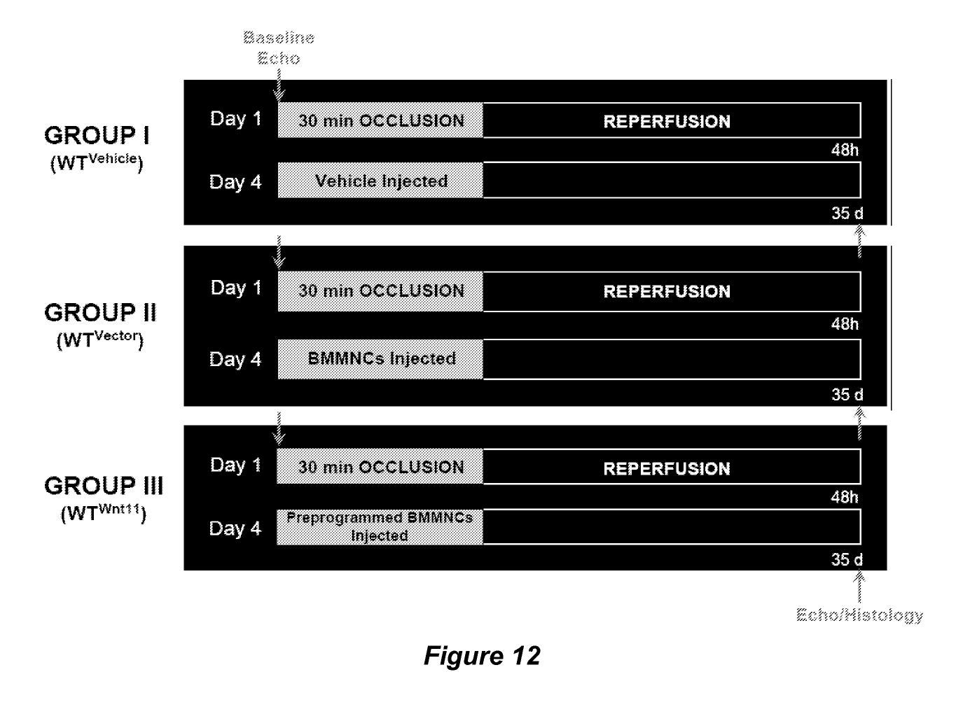

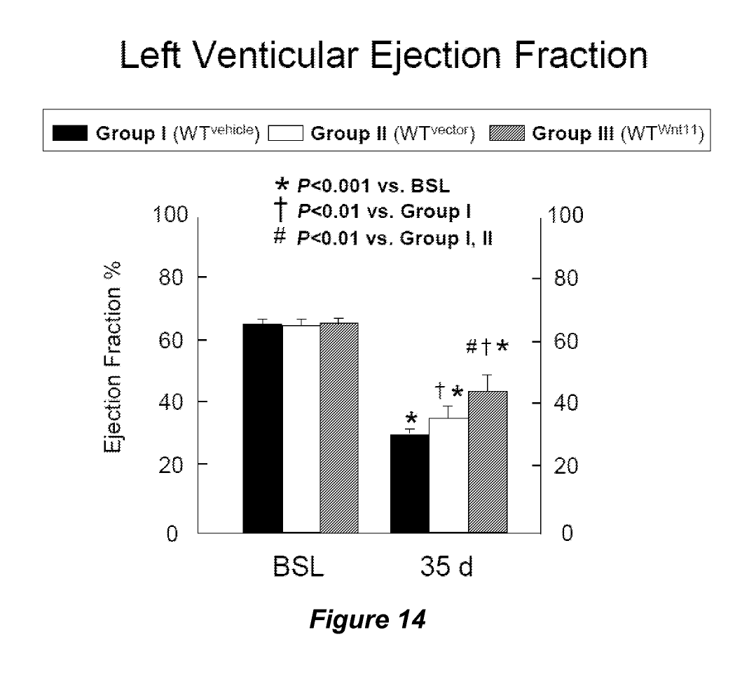

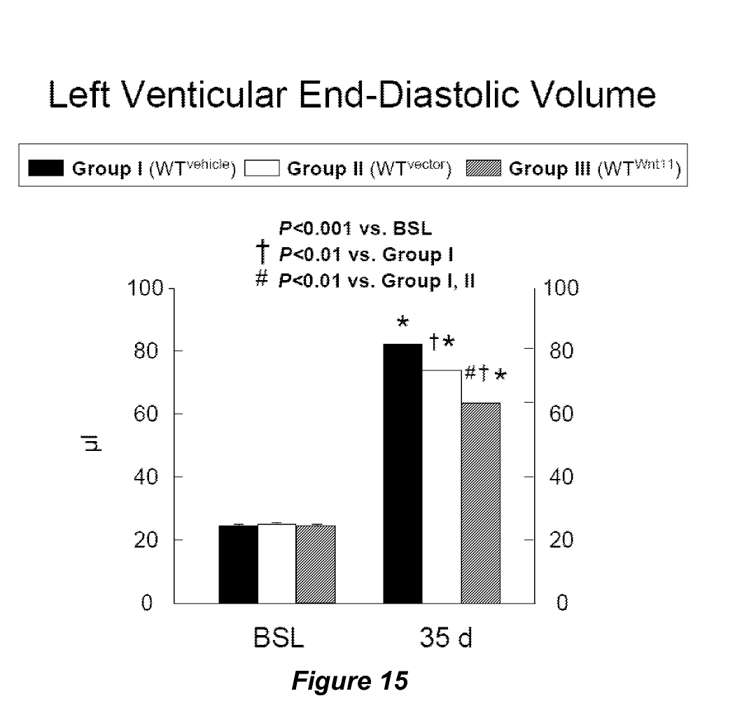

BMMNCs were isolated from C57BL/6 heterozygous mutant^{-/+} green fluorescent protein expressing (GFP^{-/+}) transgenic mice. These cells expressed GFP, and thus were easily tracked in vivo. GFP-positive BMMNCs were cultured in the presence of Wnt11 (as described hereinabove) for 3-5 days (time-point of established cardiac-specific expression; above), to induce cardiomyocytic commitment, and subsequently injected intramyocardially into the post-infarcted hearts of age-matched syngeneic homozygous negative^{-/-} transgenic siblings (not expressing GFP^{-/-}; i.e., essentially syngeneic wild type C57BL/6 mice). The experimental group was designated as group III (WT^{Wnt11}). Two additional groups of mice were studied: (i) group I, mice receiving intramyocardial injection of empty vector-treated (untreated) BMMNCs (WT^{vector}); and (ii) group II, mice receiving intramyocardial injection of vehicle or WT^{vehicle} (culture medium; see FIG. 12). To ensure in vivo trackability of BMMNCs, WT^{Wnt11} and WT^{vector} mice were euthanized and tissue analysis was performed. Cell tracking, or visualization, of successfully implanted cells was optimized via in situ immunohistochemical staining with anti-GFP antibodies. Following confirmation of intramyocardial trackability using a rat model system, all three groups of mice were subjected to echocardiographic (baseline) analysis prior to 30 minutes of ischemic injury (open-chest ligation of the left anterior descending coronary artery) and 48 hours of reperfusion. Cell therapy or vehicle was injected intramyocardilly on day 4 (48 hours following occlusion). At 35 days postinfarction echocardiographic analysis was performed (see FIG. 14).

### Example 10

### Functional Analysis of Mice Receiving Intramyocardial Injection of Wnt11-Treated BMMNCs

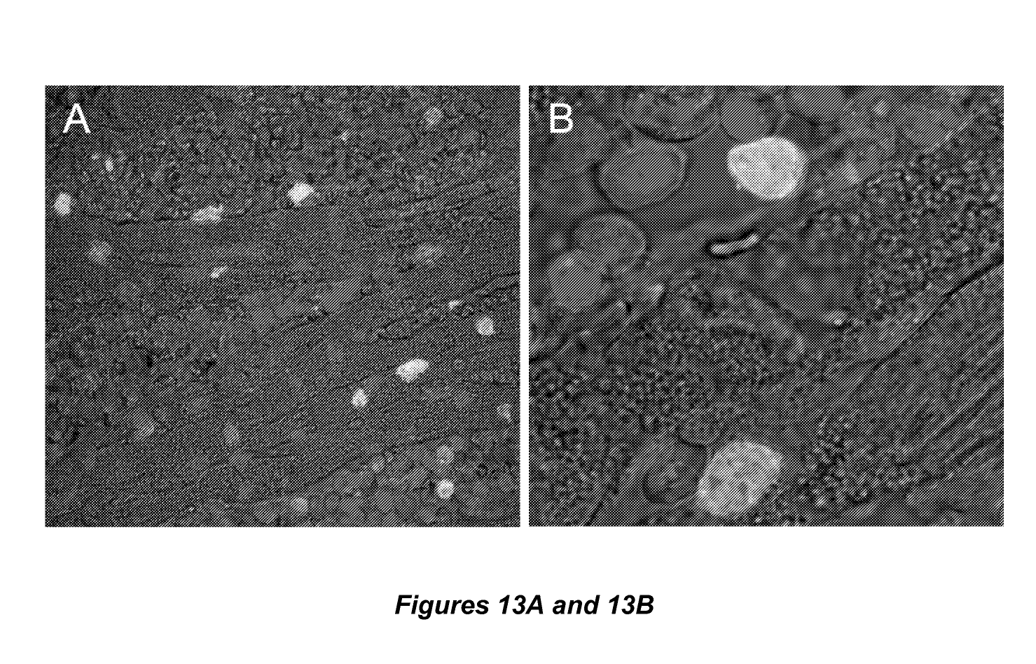

Functional analysis of mice receiving intramyocardial injection of Wnt11-treated BMMNCs was performed. As shown in FIG. 13, enhanced functional recovery (measured as left ventricular ejection fraction) of mice in group III receiving intramyocardial injection of Wnt11-treated BMMNCs (WT^{Wnt11}) compared to groups I and II was observed. In addition, group III mice display favorable left ventricular remodeling as evidenced by a reduction in left ventricular end-diastolic volume, relative to groups I and II (see FIG. 14).

### Discussion of the Examples

Although unfractionated BMMNCs are being increasingly used for cell-based cardiac repair, little is known regarding whether these cells are capable of adopting a cardiac phenotype. Disclosed herein is the development of a new approach for inducing cardiomyogenic differentiation of unfractionated BMMNCs using the non-canonical morphogen Wnt11 as the only stimulus. The disclosed results can be summarized as follows: (i) Wnt11, in itself, is sufficient to induce activation of the cardiac gene program in cultured unfractionated BMMNCs, resulting in cardiomyocytic differentiation of approximately 30% of the cells within this compartment; (ii) at the same time, Wnt11 reduces the expression of markers of pluripotency (Oct-4 and Nanog) in cultured BMMNCs; (iii) in contrast, Wnt3a upregulates markers of pluripotency, induces proliferation, and promotes hematopoietic lineage commitment; (iv) cardiomyogenic Wnt11/non-canonical signaling in BMMNCs is transduced by PKC, with JNK being a downstream mediator (but not the only one); and (v) although Wnt11 suppresses Wnt3a and β-catenin expression in unfractionated BMMNCs, inhibition of canonical signaling attenuates Wnt11/non-canonical cardiomyogenic potential, indicating cooperative synergistic cross-talk between both pathways in the determination of the differentiation fate of these cells.

This is believed to be the first study to demonstrate induction of a cardiac phenotype in unfractionated BMMNCs in vitro using a specific, well-defined, and nontoxic stimulus, without co-culture with cardiomyocytes or other cell types and without incubation with mutagens such as 5-azacytidine. In conjunction with the results disclosed herein indicating the ability of BMMNCs to undergo cardiac differentiation, the easy availability of large numbers of these cells, their beneficial effects in clinical trials, and their safety profile support the potential usefulness of BMMNCs for therapeutic cardiac repair.

The results of the presently disclosed subject matter provide the first demonstration that Wnt11, in itself, can effectively induce cardiac differentiation of unfractionated BMMNCs. The results disclosed herein suggested that Wnt11 can induce cardiac differentiation in other subsets of BMCs that are collectively isolated as BMMNCs. It is noteworthy that the Wnt11-induced expression of cardiac-specific contractile proteins and ANP in BMMNCs followed a pattern characteristic of the cardiac fetal gene program. Specifically, in addition to upregulation of ANP, BMMNCs exhibited a differential expression of the β-isoform of MyHC, which predominates in fetal life, over the α-isoform (FIG. 6), similar to the pattern observed prior to the developmental isoform-specific switch that occurs later during fetal development. This phenomenon has also been described by others in cultured embryoid bodies and MSCs (Makino et al., 1999; Auda-Boucher et al., 2000; Xu et al., 2006). The observations disclosed herein demonstrated a dichotomous effect of Wnt proteins on the expression of markers of pluripotency in unfractionated BMMNCs. The non-canonical Wnt11 reduced the expression of Oct-4 and Nanog, promoted differentiation, and had little effect on proliferation. In contrast, the canonical Wnt3a markedly increased the expression of Oct-4 and Nanog, promoted morphological homogeneity and hematopoietic potential, and enhanced proliferation. These results were consistent with previous findings regarding Wnt signaling in several other primitive cell populations, including HSCs (Reya et al., 2003; Sato et al., 2004; Nikolova et al., 2007).

The results disclosed herein demonstrated that in adult BMMNCs, PKC signaling plays a role in Wnt11-mediated JNK activation and is indispensable for the Wnt11-dependent activation of this cardiac gene program. In contrast to previous studies (Pandur et al., 2002; Garriock et al., 2005), it has been discovered that a block in JNK signaling in the Wnt11-cardiomyogenic pathway can be partially overcome, as evidenced by decreased, yet persistent, expression of Nk×2.5, GATA-4, and ANP in BMMNCs (FIG. 8). This suggested the presence of other elements responsible for Wnt11 signaling in the absence of JNK. Alternatively, the expression of GATA-4, ANP, or Nk×2.5 might not be exclusively cardiac-specific. Together with the data regarding the effect of Dkk-1, these observations suggested that although canonical signaling alone is not able to induce cardiomyogenesis in BMMNCs, it is perhaps involved in the potentiation of this effect, at least in vitro.

Summarily, disclosed herein is the discovery that adult unfractionated (density-gradient separated) BMMNCs, a cell population frequently used for cardiac repair in humans, was able to undergo cardiac differentiation in vitro in significant numbers without co-culture with cardiomyocytes. Non-canonical Wnt11 signaling via PKC is sufficient to elicit a cardiomyocytic phenotype in these cells via the induction of the cardiac fetal gene program and expression of cardiac-specific proteins. In contrast, canonical signaling via Wnt3a enhances pluripotency, promotes hematopoietic potential, and induces proliferation. These results have important implications for the understanding of Wnt signaling in adult cells as well as for therapeutic cardiac repair.

### REFERENCES

The references listed below as well as all references cited in the specification, including patents, patent applications, journal articles, and all database entries (e.g., GENBANK® Accession Nos., including any annotations presented in the GENBANK® database that are associated with the disclosed sequences), are incorporated herein by reference to the extent that they supplement, explain, provide a background for, or teach methodology, techniques, and/or compositions employed herein.

Abdel-Latif et al. (2007) Arch Intern Med 167:989-97.

Auda-Boucher et al. (2000) Dev Biol 225:214-25.

Batzer et al. (1991) Nucleic Acid Res 19:5081.

Belema Bedada et al. (2005) Mol Cell Biol 25:9509-19.

Brandon et al. (2000) Blood 96:4132-41.

Dawn & Bolli (2005) Basic Res Cardiol 100:494-503.

Eckle et al. (2006) Am J Physiol Heart Circ Physiol 291:H2533-40.

Eisenberg & Eisenberg (1999) Dev Dyn 216:45-58.

Eisenberg et al. (1997) Development 124:525-36.

Garriock et al. (2005) Dev Biol 279:179-92.

Guo et al. (1998) Am J Physiol 275:H1375-87.

Herzog et al. (2003) Blood 102:3483-93.

Koyanagi et al. (2005) J Biol Chem 280:16838-42.

Lev et al. (2005) Ann NY Acad Sci 1047:50-65.

Li et al. (2003) Circ Res 92:741-8.

Li et al. (2006) Am J Physiol Heart Circ Physiol 290:H584-9.

Makino et al. (1999) J Clin Invest 103:697-705.

Maye et al. (2004) J Biol Chem 279:24659-65.

Mitsui et al. (2003) Cell 113:631-42.

Nakamura et al. (2003) Proc Natl Acad Sci USA 100:5834-9.

Nikolova et al. (2007) Differentiation 75:100-11.

Ohtsuka et al. (1985) J Biol Chem 260:2605-2608.

Orlic et al. (2001) Nature 410:701-5.

Pandur et al. (2002) Nature 418:636-41.

Pfaffl (2001) Nucleic Acids Res 29:e45.

Reya et al. (2003) Nature 423:409-14.

Rossolini et al. (1994) Mol Cell Probes 8:91-98.

Sato et al. (2004) Nat Med 10:55-63.

Schachinger et al. (2006) N Engl J Med 355:1210-21.

Scholer et al. (1990) EMBO J 9:2185-95.

Strauer et al. (2002) Circulation 106:1913-8.

Ueno et al. (2007) Proc Natl Acad Sci USA 104:9685-90.

U.S. Pat. Nos. 5,223,409; 5,264,563; 5,498,538; 5,578,629; 5,650,489; 5,667,988; 5,702,892; 5,738,996; 5,747,334; 5,756,291; 5,780,225; 5,811,392; 5,811,512; 5,811,515; 5,817,757; 5,817,879; 5,824,483; 5,840,479; 5,858,670; 5,922,254; 5,922,545; 5,948,635; 6,057,098; 6,107,059; 6,156,511; 6,168,912; 6,174,708; 6,180,348; 6,214,553.

Xu et al. (2006) Stem Cells Dev 15:631-9.

Zuba-Surma et al. (2006) J Mol Cell Cardiol 41:650-60.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for inducing cardiomyogenic differentiation in isolated bone marrow mononuclear cells (BMMNCs), the method comprising contacting a plurality of BMMNCs with a cardiomyocyte differentiation-inducing amount of a Wnt11 gene product or a functional fragment thereof, for a time and under conditions sufficient to induce cardiomyocyte differentiation in at least a subset of the BMMNCs.

2. The method of claim 1, wherein the Wnt11 gene product comprises a Wnt11 polypeptide, or a functional fragment thereof.

3. The method of claim 2, wherein the Wnt11 polypeptide, or the functional fragment thereof, is present in a conditioned medium recovered from and/or produced by a cell culture in which at least one cell that conditioned the medium secreted the Wnt11 polypeptide or the functional fragment thereof.

4. The method of claim 1, wherein the Wnt11 gene product is a human Wnt11 gene product.

5. The method of claim 1, wherein the contacting with the Wnt11 gene product is accomplished without contacting the plurality of BMMNCs with any cells or cell types other than BMMNCs.

6. The method of claim 5, wherein the contacting occurs as the Wnt11 gene product traverses a membrane or other barrier that physically separates the BMMNCs from a source of the Wnt11 gene product or the functional fragment thereof.

7. The method of claim 6, wherein the source of the Wnt11 gene product is a cell in culture that expresses the Wnt11 gene product naturally or has been manipulated to express an endogenous or a recombinant Wnt11 coding sequence, and further wherein the endogenous or recombinant Wnt11 coding sequence encodes a Wnt11 gene product or a functional fragment thereof.

8. The method of claim 1, wherein the time and conditions sufficient to induce cardiomyocyte differentiation in at least a subset of the BMMNCs induces expression of at least one gene selected from the group consisting of cardiac troponin T (cTnT), cardiac myosin heavy chain (cMyHC)], and connexin 43.

9. A method for treating an injury to cardiac tissue in a subject, the method comprising administering to the subject a composition comprising a plurality of Wnt11-induced bone marrow mononuclear cells (BMMNCs) in a pharmaceutically acceptable carrier, in an amount and via a route sufficient to allow at least a fraction of the plurality of Wnt11-induced BMMNCs to contact the cardiac tissue, whereby the injury is treated.

10. The method of claim 9, wherein the injury is selected from the group consisting of an ischemic injury and a myocardial infarction.

11. The method of claim 9, wherein the subject is a mammal.

12. The method of claim 9, further comprising differentiating the Wnt11-induced BMMNCs to produce a plurality of cardiomyocytes or precursor cells thereof.

13. The method of claim 13, wherein the cardiomyocytes or precursor cells thereof express at least one gene selected from the group consisting of cardiac troponin T (cTnT), cardiac myosin heavy chain (cMyHC)], and connexin 43.

14. A recombinant host cell comprising an expression vector that encodes a Wnt11 polypeptide or a functional fragment thereof, optionally wherein the Wnt11 polypeptide or a functional fragment thereof is secreted from the recombinant host cell.

15. The recombinant host cell of claim 14, wherein the recombinant host cell is an isolated or immortalized human cell, optionally a human embryonic kidney-293 (HEK-293) cell.

16. The recombinant host cell of claim 14, wherein the expression vector comprises a nucleic acid sequence encoding a Wnt11 polypeptide or a functional fragment thereof operably linked to a promoter, optionally a constitutive promoter, which is active in the recombinant host cell.

17. The recombinant host cell of claim 16, wherein the Wnt11 polypeptide comprises amino acids 1-354 of GENBANK® Accession No. P51891 (quail Wnt11), or a functional fragment thereof, which is at least 95% identical at the amino acid level to amino acids 1-354 of GENBANK® Accession No. P51891, optionally over the full 354 amino acid length of GENBANK® Accession No. P51891.

18. The recombinant host cell of claim 16, wherein the Wnt11 polypeptide comprises amino acids 1-354 of GENBANK® Accession No. NP_{—}004617 (human Wnt11), or a functional fragment thereof, which is at least 95% identical at the amino acid level to amino acids 1-354 of GENBANK® Accession No. NP_{—}004617, optionally over the full 354 amino acid length of GENBANK® Accession No. NP_{—}004617.

19. A system for inducing cardiomyogenic differentiation in a cultured cell, the system comprising:
(a) a source of a Wnt11 polypeptide; and
(b) a growth area in which the cell is cultured; and optionally
(c) a barrier that physically separates the source of the Wnt11 polypeptide from the cultured cell that is permeable to the Wnt11 polypeptide, thereby allowing the Wnt11 polypeptide provided by the source to contact the cultured cell.

20. The system of claim 19, wherein the source of the Wnt11 polypeptide comprises a second cell that expresses a secretable Wnt11 polypeptide, and the barrier prevents physical contact between the second cell that expresses the secretable Wnt11 polypeptide and the cultured cell in which cardiomyogenic differentiation is to be induced.

21. The system of claim 20, wherein the second cell is a recombinant cell that comprises an expression vector encoding the secretable Wnt11 polypeptide.

22. The system of claim 21, wherein the Wnt11 polypeptide comprises an amino acid sequence selected from the group consisting of:
(a) amino acids 1-354 of GENBANK® Accession No. P51891;
(b) amino acids 1-354 of GENBANK® Accession No. NP_{—}004617;
(c) a functional fragment of (a) or (b);
(d) an amino acid sequence at least 95% identical to either (a) or (b), wherein the Wnt11 polypeptide induces cardiomyogenic differentiation in the cultured cell.
